(19) United States
(12) Patent Application Publication
(10) Pub. No.: US 2025/0177591 A1
(43) Pub. Date: Jun. 5, 2025

(54) CONTROLLING ENVIRONMENTAL CONDITIONS IN ENCLOSED SPACES

(71) Applicant: Tesla, Inc., Austin, TX (US)

(72) Inventors: Forrest Wen Liau, Austin, TX (US); Jorge C. Fialho, Austin, TX (US); Daniel F. Hanks, Austin, TX (US)

(21) Appl. No.: 18/840,410

(22) PCT Filed: Feb. 21, 2023
(86) PCT No.: PCT/US2023/013509

§ 371 Date: Aug. 21, 2024

Related U.S. Application Data
(60) Provisional application No. 63/268,427, filed on Feb. 23, 2022.

(51) Int. Cl. A61L 2/24; A61L 2/07; A61L 2/08; A61L 2/20; A61L 9/015; A61L 9/18; B60H 1/00; B60S 3/00

(52) U.S. Cl. CPC A61L 2/24; A61L 2/07; A61L 2/08; A61L 2/20; A61L 9/015; A61L 9/18; B60H 1/00878; B60S 3/008; A61L 2202/11; A61L 2202/14; A61L 2202/25; A61L 2209/111; A61L 2209/12; A61L 2209/135; A61L 2209/16

(57) ABSTRACT

The present disclosure relates to systems and methods for sanitizing an enclosed space that can be shared among multiple persons. In some embodiments, a sanitation system detects a current environmental condition associated with the enclosed space to generate sensor data. The sanitation system then obtains or generates a sanitation routine based at least in part on the sensor data. Based on the sanitation routine, the sanitation system causes a component associated with the enclosed space to adjust the current environmental condition toward a target environmental condition, where the sanitation routine specifies the component and the target environmental condition.

### CROSS-REFERENCE TO RELATED APPLICATION

This application is a non-provisional of and claims priority to U.S. Provisional Patent Application No. 63/268,427, entitled “CONTROLLING ENVIRONMENTAL CONDITIONS IN ENCLOSED SPACES,” filed on Feb. 23, 2022, which is hereby incorporated by reference in its entirety and for all purposes.

### FIELD OF THE DISCLOSURE

The present disclosure relates to a system and method for controlling environmental conditions in an enclosed space. More particularly, the present disclosure relates to a method for controlling environmental conditions related to enclosed spaces based on environmental controls at least related to light, heat, or humidity.

### BACKGROUND

Shared spaces present advantages in cost, efficiency, and environmental sustainability. For example, an automobile providing transportation throughout the day for multiple persons provides lower transportation costs and environmental footprint than an automobile used by only one person for personal commute. However, shared spaces have a disadvantage of facilitating indirect transmission of communicable diseases through contaminated air or surfaces. Current means for sanitizing shared spaces, for example manually wiping touch surfaces using disinfectant wipes, can be time-consuming, laborious and lead to unsatisfactory sanitation conditions. Additionally, such manually implemented activities may not be easily verifiable.

### BRIEF DESCRIPTION OF THE DRAWINGS

Generally described, shared spaces, such as vehicle cabins and building rooms, may require some means to sanitize the enclosed spaces. In certain scenarios, environmental conditions associated with the enclosed spaces can assist in determining the sanitation operations to be performed on the enclosed spaces.

FIG. 1 depicts an example vehicle in which embodiments of the present disclosure can be implemented.

FIG. 2 depicts an example sanitation system that can be utilized to sanitize an enclosed space, such as an interior of the vehicle of FIG. 1, in accordance with some embodiments of the present disclosure.

FIG. 3 illustrates example touch surfaces in a vehicle that can be sanitized by the sanitation system of FIG. 2 in accordance with some embodiments of the present disclosure.

FIGS. 4A-4C depicts example operations of components of a vehicle that can be caused by the sanitation system of FIG. 2 to sanitize an interior of the vehicle in accordance with aspects of the present disclosure.

FIG. 5 depicts example operations of components of a vehicle that can be caused by the sanitation system of FIG. 2 to sanitize an interior of the vehicle in accordance with aspects of the present disclosure.

FIGS. 6A-6B depict example operations of components of a vehicle that can be caused by the sanitation system of FIG. 2 to sanitize a seatbelt of the vehicle in accordance with aspects of the present disclosure.

FIGS. 7A-7C illustrate example operations of components of a vehicle that can be caused by the sanitation system of FIG. 2 to sanitize an interior of the vehicle in accordance with aspects of the present disclosure.

FIG. 8 illustrates example operations of components of a vehicle that can be caused by the sanitation system of FIG. 2 to elevate temperature within the vehicle in accordance with aspects of the present disclosure.

FIGS. 9A-9C illustrate example operations of a center display of a vehicle that can be caused by the sanitation system of FIG. 2 for reflecting sanitation lighting towards different surfaces in a vehicle cabin.

FIGS. 10A-10C illustrate example operations of service machinery performed on a vehicle that can be caused by the sanitation system of FIG. 2 for sanitizing a vehicle.

FIG. 11 illustrates an example rideshare cycle in which embodiments of the present disclosure can be implemented for sanitizing a vehicle shared by multiple riders of the vehicle.

FIG. 12 depicts a general architecture of a sanitation system for sanitizing a vehicle in accordance with aspects of the present disclosure.

FIG. 13 depicts an illustrative routine for sanitizing an enclosed space in accordance with aspects of the present disclosure.

FIG. 14A depicts a system in which environmental condition of an enclosed space can be managed in accordance with one or more aspects of the present application.

FIGS. 14B-14C depict illustrative interactions between elements of the system of FIG. 14A to manage environmental condition of an enclosed space in accordance with one or more aspects of the present application.

### SUMMARY

Provided in examples herein are systems and methods for sanitizing an enclosed space, such as a vehicle cabin and a building room that may be shared by multiple users. In some embodiments, a system for sanitizing an enclosed space is disclosed, where the system comprises a plurality of sensors and a processor. The plurality of sensors are configured to detect a current environmental condition associated with the enclosed space, and generate a first sensor data based on the current environmental condition. The processor is configured to obtain a sanitation routine or generate the sanitation routine based at least in part on the first sensor data, and execute the sanitation routine. The sanitation routine specifies at least a target environmental condition and a component associated with the enclosed space. The sanitation routine, when executed by the processor, causes the component to adjust the current environmental condition toward the target environmental condition.

In some embodiments, a computer-implemented method for sanitizing an enclosed space that comprises a plurality of sensors and a processor is disclosed. The method comprises: (1) detecting, by the plurality of sensors, a current environmental condition associated with the enclosed space; (2) generating, by the plurality of sensors, a first sensor data based on the current environmental condition; (3) receiving or generating, by the processor, a sanitation routine, wherein the sanitation routine is generated based at least in part on the first sensor data; and (4) causing, by the processor, a component associated with the enclosed space to adjust the current environmental condition associated with the enclosed space toward a target environmental condition, wherein the sanitation routine specifies at least the target environmental condition and the component associated with the enclosed space.

In some embodiments, one or more non-transitory computer-readable media comprising instructions executable at a sanitation system of an enclosed space is disclosed. When executed by the sanitation system, the instructions cause the sanitation system to: (1) detect a current environmental condition associated with the enclosed space; (2) generate a first sensor data based on the current environmental condition; (3) generate a sanitation routine based at least in part on the first sensor data; and (4) execute the sanitation routine, wherein the sanitation routine specifies at least a target environmental condition and a component associated with the enclosed space, and wherein executing the sanitation routine causes the component to adjust the current environmental condition toward the target environmental condition.

### DETAILED DESCRIPTION

Generally described, one or more aspects of the present disclosure correspond to systems and methods for controlling environmental conditions in enclosed spaces. Illustratively, aspects of the present disclosure relate to control of environment conditions in enclosed spaces for purposes of sanitizing the enclosed space. Illustratively, the present disclosure can include a combination of mechanisms to inspect the enclosed space, mechanisms to determine environmental conditions, and mechanisms to adjust the condition within the enclosed space. The various settings or attributes of the environmental conditions may be configured based on settings/levels selected to deactivate pathogens. The enclosed space may be inside a vehicle containing sensors capable of determining the presence of humans in a cabin, sensors capable of determining cabin temperature, and a data network capable of retrieving information about surrounding environmental conditions. The cabin may also include a heating, ventilation, and air conditioning (HVAC) system capable of accumulating moisture, raising cabin temperature, and directing heated air towards particular touch surfaces.

In another embodiment, the vehicle cabin contains active heating elements and materials that passively emit far infrared radiation behind the touch surfaces. In another embodiment, the vehicle cabin contains lights providing specific wavelengths for disinfection. In these embodiments, the disinfection process occurs only after determining that humans or other sensitive entities such as pets, groceries, medicine, or portable electronics are not present in the vehicle. In some embodiments, a mechatronic system in the enclosed space operates together with the sanitation mechanism for more effective cleaning.

Specifically, aspects of the present disclosure relate to a sanitation system that can be utilized to sanitize an enclosed space, such as a cabin of a vehicle. Illustratively, the sanitation system can include a plurality of sensors and a processor in communication with the plurality of sensors. The plurality of sensors can be configured to detect the absence of one or more entities (e.g., humans, pets, groceries, medicines, or electronic devices) within the enclosed space. In detecting the absence of one or more entities, the plurality of sensors may generate first sensor data. The plurality of sensors can further be configured to detect a plurality of environmental conditions associated with the enclosed space. The environmental conditions can be conditions within the enclosed space (e.g., temperature and humidity within the enclosed space) and/or conditions external to the enclosed space (e.g., weather outside the enclosed space). In detecting the plurality of environmental conditions, the plurality of sensors may generate a second sensor data. Based on the first sensor data generated by the plurality of sensors, the processor may then determine the absence of one or more entities within the enclosed space. In some embodiments, in response to determining that the one or more entities within the enclosed space is absent, the processor may generate a sanitation routine based on the second sensor data. The sanitation routine may include emitting disinfection vapor to sanitize the enclosed space, adjusting positions of seats within the enclosed space to allow more exposure to light irradiation and the like. The processor may then cause one or more component(s) associated with the enclosed space to implement the sanitation routine. For example, the processor may cause an autonomous driving system of a vehicle to orient the vehicle in a particular direction to expose more touch surfaces within the vehicle to sunlight.

In some embodiments, a sanitation system detects a current environmental condition associated with the enclosed space to generate sensor data. The sanitation system then obtains or generates a sanitation routine based at least in part on the sensor data. Based on the sanitation routine, the sanitation system causes a component associated with the enclosed space to adjust the current environmental condition toward a target environmental condition, where the sanitation routine specifies the component and the target environmental condition.

Generally described, sanitation of an enclosed space, such as a vehicle passenger compartment and a hotel room, that may be occupied by different people is of high importance. In the setting of a vehicle cabin, especially those in shared vehicles, sanitation and disinfection between users is desired. Although shared spaces present advantage in cost, efficiency and environmental sustainability, shares spaces present a disadvantage of increased rates of soiling and the facilitation of indirect transmission of communicable diseases through contaminated air or surfaces. Current means for sanitizing shared spaces, such as manually wiping touch surfaces using disinfectant wipes, can be time-consuming, laborious and untimely. Furthermore, current means for sanitizing an enclosed space might lead to ineffective sanitation because of the presence of blind spots or area of an enclosed space that is difficult to be reached.

To address at least some of the above problems, systems and methods that are capable of sanitizing an enclosed space without manual sanitation are implemented by the coordination between a sanitation system and components associated with the enclosed space. More specifically, the sanitation system may be capable of automatically inspecting the enclosed space, determining environmental conditions associated with the enclosed space, and causing components associated with the enclosed space to sanitize the enclosed space to deactivate pathogens based on the determined environmental conditions. The sanitation system may include sensors that can detect presence of sensitive entities such as humans within the enclosed space. The sanitation system may further include sensors that can detect environmental conditions of an enclosed space, such as the temperature or humidity within the enclosed space. The sanitation system may further include a network module or be in communication with a network module separate from the sanitation system to retrieve information about environmental conditions surrounding the enclosed space, such as the weather at the moment. Based on the detected environmental conditions, the sanitation system may cause different components associated with the enclosed space to perform operations that can be utilized to sanitize the enclosed space. For example, the sanitation system may cause a heating, ventilation and air conditioning (HVAC) system of a vehicle to perform operations for sanitizing the vehicle, including accumulating moisture, raising vehicle cabin temperature and directing heated air towards particular touch surfaces within the cabin of the vehicle based on the current temperature and humidity within the vehicle.

In some embodiments, the sanitation system may employ sensors that are capable of detecting presence of humans, pets and certain objects in a vehicle. In addition to temperature and humidity within the vehicle cabin, the sensors may further detect air particulates, pathogens, soiling and sounds and the like within the vehicle cabin. In some embodiments, when a particular pathogen is detected at certain touch surfaces, the sanitation system may direct specific disinfection light to be emitted toward the touch surfaces where the particular pathogen is detected. In addition to adjusting temperature or humidity within the vehicle cabin, the operations employed by the sanitation system may include moving or re-orienting the vehicle to allow more exposure of the vehicle cabin to sunlight. In some embodiments, the sanitation operations performed may be dependent upon the detected environmental conditions. For example, when the sanitation system determines that there is sufficient sunlight outside the vehicle cabin based on sensor data generated by the sensors, the sanitation system may cause a door window or a roof window of the vehicle to be opened to allow more penetration of sunlight to sanitize the vehicle cabin. In contrast, when the sanitation system determines that it is raining outside based on sensor data generated by the sensors, the sanitation system may cause a light source within the vehicle cabin to irradiate disinfection light to sanitize the vehicle cabin.

In some embodiment, the sanitation system may cause an autonomous driving system of a vehicle to park the vehicle near a non-mobile cleaning robot for sanitizing the vehicle cabin. The sanitation system may further trigger the opening of a window of the vehicle to allow the cleaning robot to more effectively sanitize the vehicle cabin. In some embodiments, processor(s) within the sanitation system may perform handshake protocols with the cleaning robot through a network module of the sanitation system to negotiate the time duration and specific operations (e.g., infrared lighting, contact heating or steam sanitation) the cleaning robot is to perform on the vehicle. In some embodiments, prior to implement a sanitation routine, one or more processors of the sanitation system may determine whether there are any sensitive entities such humans or pets inside the vehicle cabin. In response to determining that there are no sensitive entities inside the vehicle cabin, the sanitation system may cause a component associated with the enclosed space to implement the sanitation routine.

Although the various aspects will be described in accordance with illustrative embodiments and combination of features, one skilled in the relevant art will appreciate that the examples and combination of features are illustrative in nature and should not be construed as limiting. More specifically, aspects of the present application may be applicable with various types of enclosed spaces, sanitation equipment (e.g., disinfection light sources or vaporizers), interfaces and the like. Still further, although a specific sanitation procedure for automatically sanitizing an enclosed space will be described, such illustrative sanitation procedure should not be construed as limiting. Accordingly, one skilled in the relevant art will appreciate that the aspects of the present application are not necessarily limited to application to any particular type of enclosed space, sanitation equipment, and/or sanitation methodology.

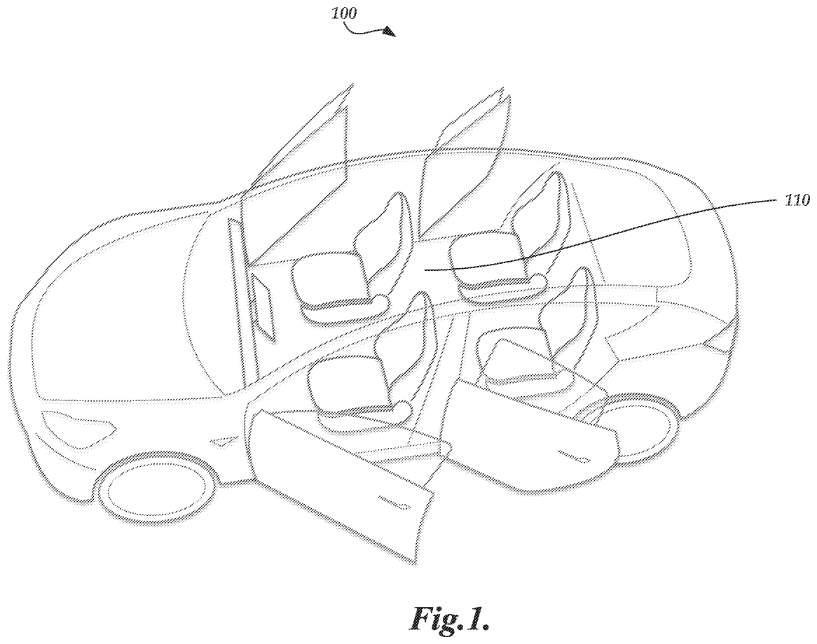

FIG. 1 depicts an example vehicle 100 having an enclosed space 110 in which embodiments of the present disclosure can be implemented. Although an automobile is shown in FIG. 1 as the example vehicle 100, the vehicle 100 can be different types of transportation tools, including but not limited to aircraft, spacecraft, trucks, vessels, maritime, ferry and vans. Alternatively, the vehicle 100 may be an automobile shared by different users throughout the day. As shown in FIG. 1, the enclosed space 110 is an interior or passenger compartment of the vehicle 100. In other embodiments, enclosed spaces with which embodiments of the present disclosure can be implemented include rooms inside static buildings such as houses, apartments, offices, hospitals, factories or the like. The enclosed space 110 may be equipped with a sanitation system (not shown in FIG. 1) capable of determining environmental conditions including sensing temperature, humidity, sunlight intensity, and presence of airborne pathogens, inside or outside of the enclosed space 110. As will be described in greater detail below, the sanitation system can include a plurality of sensors and a processor; the sanitation system may further include or work in conjunction with other subsystems, such as lighting control subsystems, humidity control subsystems, and autonomous driving subsystems of a vehicle.

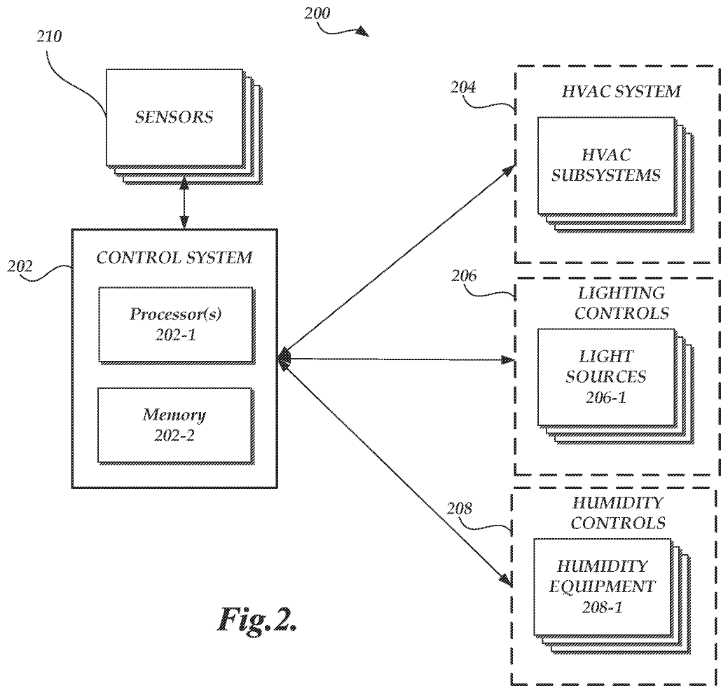

FIG. 2 illustrates an example sanitation system 200 that can be deployed within an enclosed space of a vehicle or building room, such as the enclosed space 110 in FIG. 1, in accordance with some embodiments of the present disclosure. As shown in FIG. 2, the sanitation system 200 includes a plurality of sensors 210 and a control system 202 that has at least a processor 202-1 and a memory 202-2. The sanitation system 200 may further include a HVAC system 204, a lighting control 206, and a humidity control 208. In some embodiments, the HVAC system 204, the lighting control 206 and the humidity control 208 are existing components associated with a vehicle, such as an electric vehicle, and can be utilized by the control system 202 for sanitation purposes. For example, the HVAC system 204 may be the heat, ventilation and air conditioning system of an electric vehicle and the control system 202 can be in communication with the HVAC system 204 of the electric vehicle to utilize or cause the HVAC system 204 to sanitize the interior of the electric vehicle. As another example, the light sources 206-1 may be a light source that has already been installed on a vehicle, such as a LED light of the vehicle. The light sources 206-1 may be deployed in different parts of the cabin of the vehicle to increase intensity of lighting or exposure to the lighting that may lead to better sanitation. For example, a light source may be deployed in the front cabin, another light source may be deployed in the middle of the cabin and still another light source may be deployed in the back of the cabin. In some embodiments, the sanitation system 200 can coordinate several components or systems (e.g., an autonomous driving system or mechatronics components or wireless networking interfaces) of a vehicle for sanitizing the interior of the vehicle. For example, the sanitation system 200 can cause a seatback of a seat recline and control the LED light of the vehicle to emit light toward certain touch surfaces that are exposed to the LED lighting after the seatback is reclined. By utilizing components that exist already, the sanitation system 200 can sanitize an enclosed space at no or moderate cost overhead.

The plurality of sensors 210 may include vision sensor(s) (e.g., for analyzing images from interior or exterior of an enclosed space), acoustic sensor(s), thermal sensor(s), weight sensor(s), pressure sensor(s), capacitive sensor(s), radio frequency sensor(s) (e.g., for detecting human body movement, breathing, or heartbeat), laser sensor(s), humidity sensor(s) or gas detectors. The plurality of sensors 210 may be used to generate sensor data for determining occupancy of an enclosed space. For example, the plurality of sensors 210 may include a camera that captures images in the enclosed space and the captured images may be provided to the processor(s) 202-1 to analyze whether there is presence of humans or other sensitive entities such as pets, medicine or electronic devices. In some examples, some of the sensors may be deployed at the same spot of the enclosed space. For instance, the plurality of sensors 210 may include a humidity sensor and a temperature sensor that are integrated and installed around the windshield or other spots of a vehicle.

In some embodiments, the plurality of sensors 210 may also generate sensor data that may be used to indirectly infer occupancy of an enclosed space. For example, the plurality of sensors 210 may include a motion sensor that is attached to a door of a vehicle to generate motion data that is indicative of opening and closing of the door. The motion data can be transmitted to the processor(s) 202-1 to infer occupancy of the vehicle based on the door opening and closing history. In some embodiments, the plurality of sensors 210 may be integrated with a user interface associated with an enclosed space to collect sensor data to determine whether sensitive entities are in the enclosed space. For example, an user interface may ask an user to indicate whether heat-sensitive entities are present in the enclosed space and an acoustic sensor (e.g., for detecting human voices) or a capacitive sensor (e.g., installed within a touch control panel of the user interface) can be used to capture the response of the user for the processor(s) 202-1 to determine whether heat-sensitive entities are present.

Illustratively, to adjust environmental or sanitation conditions (e.g., deactivate pathogens) within an enclosed space, an enclosed space may utilize its heating, ventilation, and air conditioning (HVAC) system 204 to elevate air temperature. For example, the enclosed space may be a cabin of a vehicle and the HVAC system 204 may be the HVAC system equipped in the vehicle. As such, the processor(s) 202-1 may activate the HVAC system 204 to increase temperature within the cabin of the vehicle. The sanitation system 200 can further employ humidity controls 208 to adjust humidity of an interior of a vehicle, and employ lighting controls 206 to utilize lights of specific wavelengths to disinfect surfaces of the interior of the vehicle. For example, humidity controls 208 may include a humidity equipment 208-1 such as an evaporator that can accumulate moisture from external air and then switch to recirculation mode for circulating the accumulated moisture around the passenger compartment of a vehicle. In some examples, the evaporator may further capture moisture from an occupant's breath and then release the moisture back into the enclosed space when the passenger compartment of the vehicle is unoccupied. In other examples, the evaporator can remove condensation from the interior surface of windows of the vehicle by emitting heated air channeled towards the windows. In still other examples, the humidity equipment 208-1 may include a dehumidifier that can remove moisture from the enclosed space to reach a target relative humidity level. Although the HVAC system 204, humidity controls 208, lighting controls 206 and the plurality of sensors 210 are illustrated as separate subsystems in FIG. 2, in some embodiments, parts or all of these subsystems may be combined as an integrated environmental control system that is deployed within an enclosed space (e.g., a cabin of a vehicle). For example, the sensors 210 may include a humidity sensor that is a part of the humidity equipment 208-1 and when the humidity sensor detects that the humidity of an enclosed space is at certain level, the processor(s) 202-1 may trigger the humidity equipment 208-1 to increase or decrease the humidity of the enclosed space based on the reading of the humidity sensor that is integrated with the humidity equipment 208-1. In still other embodiments, the humidity equipment 208-1 may include an ultrasonic vaporizer, a steam vaporizer, a mist sprayer, a capillary evaporator, an impeller vaporizer, a water dripper or the like, and the control system 202 may cause the humidity equipment 208-1 to spray vapor based on environmental conditions detected by the sensors 210.

As described above, one or more aspects of the present application correspond to a set of processes for controlling environmental conditions for exposing touch surfaces of an enclosed space, such as the enclosed space 110 of the vehicle 100 in FIG. 1. Illustratively, the environmental conditions can include control of temperature, humidity and light radiation such that possible contaminants on the touch surfaces will be reduced or eliminated. In some embodiments, a combination of humidity, temperature and light radiation can result in a reduced number of antigens or other contaminants on touch surfaces or within the air of the interior compartment. In some embodiments, the HVAC system 204, the lighting controls 206 and the humidity controls 208 are configured by the control system 202 to reach a target combination of humidity, temperature and light radiation to inhibit a particular type of bacteria or virus. For example, research statistics show that the half-life of SARS-COV-2 would be around eighteen hours when the temperature is between 70° F. to 75° F., the humidity is 20% and there is no light radiation. The half-life of the SARS-COV-2 would decrease to around six hours when the temperature is between 70° F. to 75° F., there is no light radiation, and the humidity is increased to 80%. The half-life of the SARS-COV-2 would further decrease to around two minutes when the temperature is between 70° F. to 75° F., the humidity is 80%, and there is light radiation. As such, the control system 202 may control the HVAC system 204 to adjust the temperature to between 70° F. to 75° F., prompt the lighting controls 206 to turn on light sources 206-1 to irradiate light, and control the humidity controls 208 such that the humidity equipment 208-1 adjusts the humidity to 80% to inhibit the spread or eliminate the SARS-COV-2 from touch surfaces or interior of an enclosed space. Other research statistics associated with other pathogens can also be loaded into the memory 202-2 of the control system 202 to enable the processor(s) 202-1 to configure the HVAC system 204, the lighting controls 206 and the humidity controls 208 to target environmental or sanitation conditions to inhibit the spread or growth of these pathogens in an enclosed space. Although not shown in FIG. 2, the sanitation system 200 can further direct a component associated with an enclosed space for sanitation purpose. More specifically, the sanitation system 200 may control the movement of a seatbelt, seatback, a glovebox or other components of a vehicle for performing different sanitation or hygiene operations. These operations will be described in greater detail below.

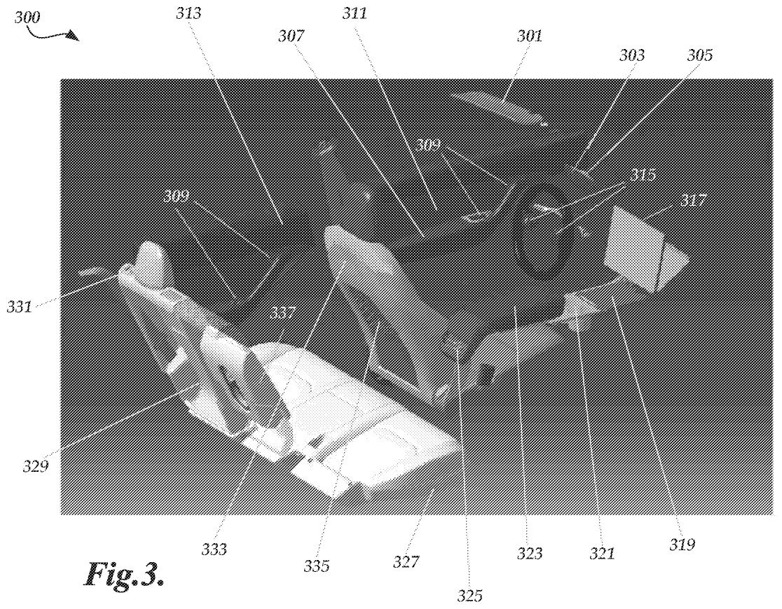

FIG. 3 illustrates example touch surfaces inside an enclosed space 300 in which embodiments of the present disclosure can be implemented. As shown in FIG. 3, example touch surfaces include overhead visor 301, mirror 303, and mirror housing 305; door armrest 307, switches 309, insert panel 311, and belt line panel 313; steering wheel with finger controls 315; center touch display 317, console lid 319, cupholders 321, armrest 323, and vent panel 325; and seat cushion 327, seatback 329, headrest 331, back panel 333, storage pocket 335, and folding armrest 337. In some embodiments, sanitation can be directed by a sanitation system, such as the sanitation system 200 of FIG. 2, toward touch surface(s) that has been recently touched or is most frequently touched according to sensor data. For example, the plurality of sensors 210 may include a touch sensor (e.g., capacitive touch sensor) that is installed on the back panel 333. When the back panel 333 is touched, the sensors 210 would generate sensor data indicating a touch event on the back panel 333. The sensor data would be transmitted to the processor(s) 202-1 for determining whether sanitation on the back panel 333 is needed. For example, when the occurrence of the touch events exceeds certain frequency or number of times (e.g., the back panel 333 has been touched more than ten times), the processor(s) 202-1 may determine sanitation on the back panel 333 is necessary and may prompt the light sources 206-1 to direct irradiation light toward the back panel 333. The light sources 206-1 may be configured to emit visible light, invisible light, infrared light (e.g., near-infrared and/or far-infrared) and/or ultraviolet light. In some embodiments, the processor(s) 202-1 would direct the irradiation light toward the back panel 333 after determining that no sensitive entities are present within an enclosed space. Alternatively, the processor(s) 202-1 may prompt the humidity equipment 208-1 (e.g., a vaporizer) to emit disinfection vapor toward the back panel 333. Advantageously, sanitation can be performed directed towards surfaces that are touched more often and be less performed on rarely touched surfaces to save resources and energy.

Additionally, other touch surfaces within a vehicle that are not explicitly shown in FIG. 3 can also be sanitized by a sanitation system in accordance with embodiments of the present disclosure, such as the sanitation system 200 of FIG. 2. Other touch surfaces not shown in FIG. 3 include, but not limited to, control stalks, overhead switches, seat side shield and restraint system including seatbelt, latch plate, buckle, and the like. In addition to emitting light and disinfection vapor toward the touch surfaces, the sanitation system 200 may direct the HVAC system 204 to adjust the temperature and/or humidity within an enclosed space to a certain level. More specifically, the sanitation system 200 may cause the HVAC system 204 to elevate the temperature in the enclosed space to around 56° C. for thirty minutes to eliminate the SARS-COV-2.

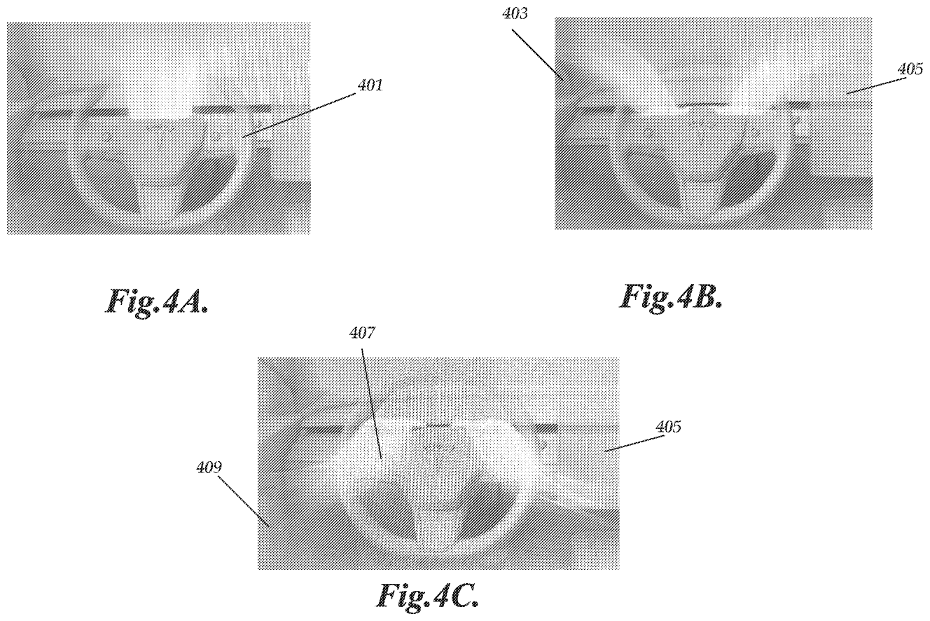

FIGS. 4A-4C illustrate various embodiments of operations of the sanitation system 200 of FIG. 2 for sanitizing an enclosed space. More specifically, FIG. 4A illustrates the operation of the HVAC system 204, where the HVAC system 204 blow heated air toward the top of a steering wheel 401. The operation of FIG. 4A may be triggered after the plurality of sensors 210 (e.g., an acoustic sensor or a pressure sensor) detect an event where a driver coughs or sneezes toward the top of the steering wheel 401. More specifically, after such an event is detected, the processor(s) 202-1 can prompt and configure the HVAC system 204 to blow heated air toward the top of the steering wheel 401. Alternatively, a humidity equipment 208-1 can be employed to evaporate disinfection vapor toward the top of the steering wheel 401. FIG. 4B illustrates the operation of the HVAC system 204, where the HVAC system 204 blow heated air towards a door window 403 and a top portion of a center display 405. The operation of FIG. 4B may be triggered after the plurality of sensors 210 (e.g., a capacitive touch sensor) detect that the door window 403 and/or the top portion of the center display 405 has been touched by a user. More specifically, based on sensor data provided by the plurality of sensors 210, the processor(s) 202-1 may determine that the door window 403 and/or the top portion of the center display 405 has been touched over a threshold amount of times (e.g., five times) and may generate a sanitation routine that directs the HVAC system 204 to blow heated air towards the door window 403 and/or the top portion of the center display 405. FIG. 4C illustrates the operation of the HVAC system 204, where the HVAC system 204 blow heated air directed towards steering wheel finger controls 407, door armrest 409, and center display 405. The operation of FIG. 4C may be triggered after the plurality of sensors 210 (e.g., a vision sensor) show that the door armrest 409 has been touched. For example, the vision sensor may capture images showing that a user has touched the door armrest 409. The processor(s) 202-1 may then analyze the captured images to determine the door armrest 409 has been touched. The processor(s) 202-1 may further determine that the enclosed space is not currently occupied before generating a sanitation routine that would then trigger the HVAC system 204 to blow heated air towards the door armrest 409.

In some embodiments, the processor(s) 202-1 may cause the HVAC system 204 of the vehicle to enter a vapor removal mode to remove vapor from an occupant's mouth when the processor(s) 202-1 determines a cough or sneeze just occurred. For example, while there is still occupant inside the vehicle, the processor(s) 202-1 may cause the HVAC system 204 to transport vapor from an occupant's mouth to external of the vehicle based on sensor data (e.g., image data or acoustic data) generated by the plurality of sensors 210 indicating that an occupant just sneezed or coughed. Advantageously, the spread of pathogens may be reduced by detecting vapor from an occupant and removing the vapor at an earlier time (e.g., compared with sanitizing in response to detecting no occupant inside the vehicle).

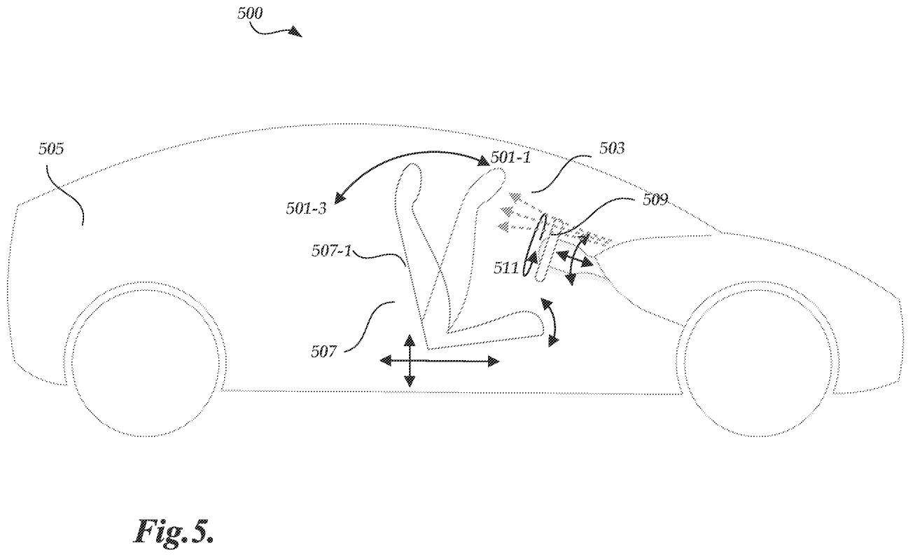

FIG. 5 depicts example operations of components of a vehicle 500 that can be caused by a sanitation system, such as the sanitation system 200 of FIG. 2, to sanitize an interior of the vehicle. Illustratively, the sanitation system 200 can operate in coordination with components of a vehicle to sanitize an enclosed space. The components of the vehicle may include seating components (e.g., seat and/or seatbelt), arm rests, storage compartments and the like. FIG. 5 illustrates altering the positions and orientations of a seat 507 and a steering wheel 509 to expose different parts of the seat and the steering wheel to disinfection lighting, vapor or heated air. The operations in FIG. 5 may be triggered by the processor(s) 202-1 when the processor(s) 202-1 determines, based on sensor data provided by the plurality of sensors 210, that sensitive entities are not within the enclosed space. More specifically, based on sensor data provided by the plurality of sensors 210, the processor(s) 202-1 may further determine that the back 505 of the vehicle needs to be sanitized. The processor(s) 202-1 may then prompt the seat 507 to recline the seatback 507-1 to backward from position 501-1 to 501-3 so that heated air (or disinfection vapor) 503 may have an air path to the back 505 of the vehicle without being obstructed by the seatback 507-1. In some embodiments, the seat 507 may be moved forward or backward to expose some unexposed area to disinfection lighting. For example, the seat 507 can be moved backward so that the light sources 206-1 that might be on top of the seat 507 can irradiate light directed toward area that was covered by the seat 507. As illustrated in FIG. 5, the steering wheel 509 can be elevated or be oriented differently to allow exposure of area that otherwise would be blocked from sanitizing light and/or vapor. For example, the steering wheel 509 may be steered in a counterclockwise direction 511 at a certain pace (e.g., steering 360 degrees in sixty seconds) to effectively allow different parts of the steering wheel 509 be exposed to the heated air (or disinfection vapor) 503.

Additionally, the processor(s) 202-1 may adjust the movements of the components associated with the enclosed space. More specifically, the processor(s) 202-1 may adjust the movement of the seat 507 forward or backward in incremental manner (e.g., moving backward 5 cm every 10 seconds) or along a defined progression of movements (e.g., moving backward after moving forward, followed by moving upwards and downwards). In other embodiments, although not shown in FIG. 5, the sanitation system 200 can also trigger the exposure of interior surfaces of an enclosed space, such as glovebox component in the interior of a vehicle. For example, based on sensor data provided by the plurality of sensors 210 (e.g., a capacitive touch sensor), the processor(s) 202-1 may determine that an interior of a glovebox in a front passenger seat has been touched. The processor(s) 202-1 may then send control signal to a mechatronic component of the glovebox to open the glovebox such that the interior of the glovebox can be exposed to disinfection light, heated air or sanitizing vapor.

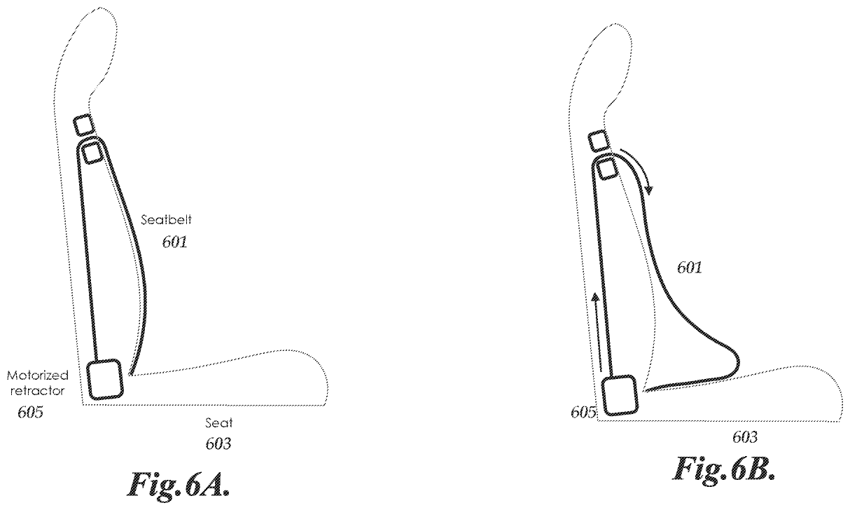

FIGS. 6A and 6B depict example operations of a seatbelt 601 in a vehicle that can be caused by a sanitation system, such as the sanitation system 200 of FIG. 2, in accordance with embodiments of the present disclosure. FIG. 6A illustrates a seatbelt 601 of a seat 603 in an unused condition. As indicated in FIG. 6A, some portions of the seatbelt 601 are hidden inside the motorized retractor 605 due to the seatbelt 601 being in the unused condition. As such, heated air, disinfection light or vapor may not effectively reach those hidden portions of the seatbelt 601. FIG. 6B illustrates the use of the motorized retractor 605 to extend the seatbelt 601 so as to increase exposure of the seatbelt 601 to sanitation. More specifically, the processor(s) 202-1 may determine that the seatbelt 601 has been used and touched based on sensor data generated by the plurality of sensors 210. The processor(s) 202-1 may further determine that the vehicle is unoccupied based on the other sensor data generated by the plurality of sensors 210. The processor(s) 202-1 may then cause the motorized retractor 605 to roll out portions of the seatbelt 601 that were hidden inside the motorized retractor and cause the HVAC system of the vehicle to release heated air directed toward the seatbelt 601 of the seat 603 for sanitation. Alternatively, the processor(s) 202-1 may cause the light sources 206-1 to irradiate disinfection light toward the seatbelt 601 or cause the humidity equipment 208-1 (e.g., a vaporizer) to release disinfection vapor toward the seatbelt 601. In some embodiments, the processor(s) 202-1 may set a time duration for sanitizing the seatbelt 601. For example, after sanitizing the seatbelt 601 for certain amount of time (e.g., thirty seconds), the processor(s) 202-1 may cause the light irradiation, vapor or heat emission to stop and cause the motorized retractor 605 to roll back the seatbelt 601 such that the seatbelt 601 may return back to the unused condition as illustrated in FIG. 6A.

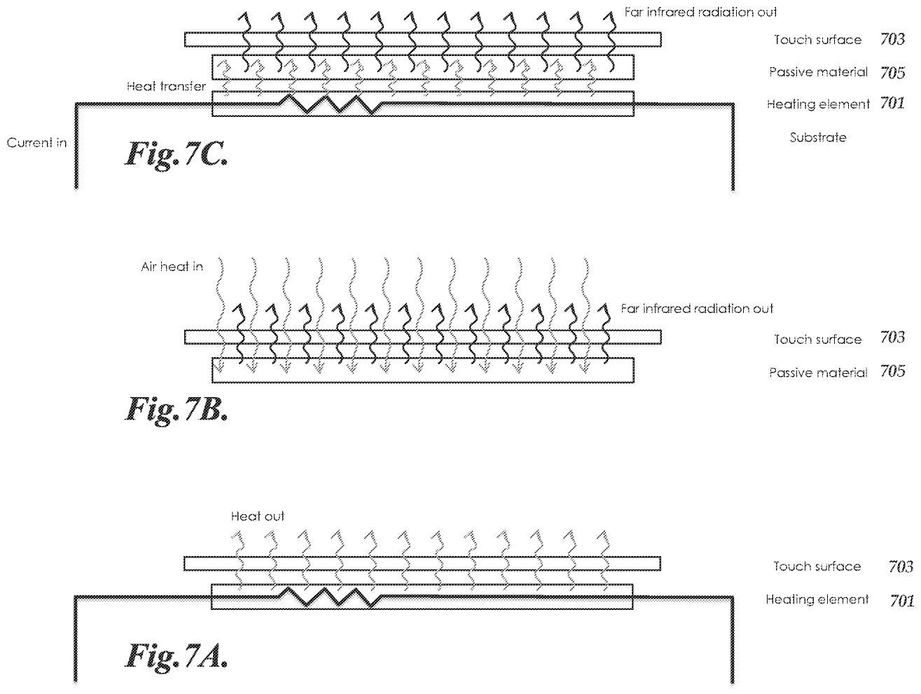

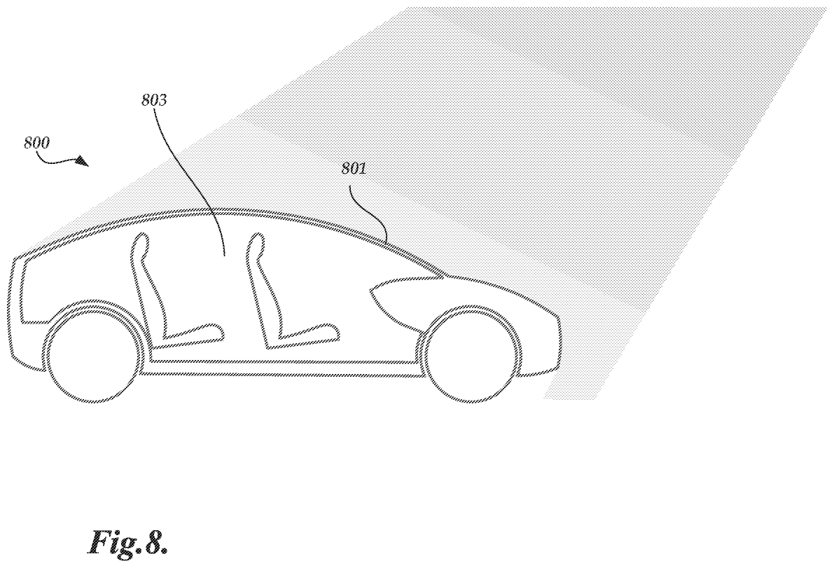

FIGS. 7A-7C illustrate example components that may be utilized by a sanitation system, such as the sanitation system 200 in FIG. 2, to provide heat for sanitizing an enclosed space. In some embodiments, the components shown in FIGS. 7A-7C may be integrated as a part of the HVAC system 204 of FIG. 2. In other embodiments, the components shown in FIGS. 7A-7C may be separate components that are not parts of the HVAC system 204 of FIG. 2. As shown in FIG. 7A, an active component (i.e., a heating element 701) serves as a heat source and conducts heat toward a touch surface 703. The touch surface 703 may be any one of the touch surfaces depicted in FIG. 3 or other touch surfaces not depicted in FIG. 3. The heating element 701 may be one or more of insulated wires, printed circuits, positive temperature coefficient (PTC) films, conductive textiles, or other conductive films. In some embodiments, the heating element 701 may be turned off (e.g., providing no electrical power) by the processor(s) 202-1 when the processor(s) determines that an enclosed space is occupied by sensitive entities based on sensor data provided by the plurality of sensors 210. When the processor(s) determines that the touch surface 703 of FIG. 7A has been touched based on other sensor data provided by the plurality of sensors 210 and that the enclosed space is not occupied by sensitive entities, the processor(s) 202-1 may cause (e.g., by providing electrical power) the heating element 701 to emit heat toward the touch surface 703.

In other embodiments, passive elements can be utilized to provide heat to an enclosed space for sanitation purposes. FIG. 7B illustrates using a passive material 705 to provide heat to an enclosed space, such as the enclosed space 110 of FIG. 1. The passive material 705 may be incorporated into an interior of a vehicle, such as a compartment or a cabin of a vehicle. Illustratively, the passive material 705 may obtain energy from environmental inputs, such as air, sunlight, or the like. As shown in FIG. 7B, the passive material 705 emits far infrared radiation toward a touch surface 703 when the passive material 705 receives energy from energy sources such as sunlight. The passive material 705 may be a ceramic material deposited on beneath a surface material of a touch surface 703 or embedded within a surface material of the touch surface 703. In some embodiments, heating elements and passive material can be combined to provide heat to a touch surface of an enclosed space for sanitation purposes. FIG. 7C illustrates combining a heating element 701 and a passive material 705 to sanitize a touch surface 703. As shown in FIG. 7C, the heating element 701 generates heat, which is conducted to the touch surface 703. In addition, the passive material 705 can receive the heat from the beating element 701 and radiates far infrared radiation toward the touch surface 703. As such, the heat directed toward the touch surface 703 may be increased compared with the use of only heating elements 701 or passive materials 705. Advantageously, the increased heat may result in better sanitizing the touch surface 703.

Illustratively, an enclosed space in which embodiments of the present disclosure can be implemented may be an interior of a vehicle. In some embodiments, a sanitation system (such as the sanitation system 200 of FIG. 2) may be deployed within a vehicle and the sanitation system may utilize navigational and directional systems or vision systems of the vehicle to make use of various external environmental contributions as part of control environmental factors. More specifically, in some embodiments, the sanitation system 200 may cause navigational, directional, vision systems, and the like of a vehicle to identify opportunities for use of light energy as part of the management of environmental conditions within the internal compartment of the vehicle. For example, the sanitation system 200 may cause an autonomous driving system of the vehicle to orient the vehicle for receiving detected solar energy. In another example, the sanitation system 200 may cause the autonomous driving system of the vehicle to select navigational paths (e.g., specific roads or lanes of roads) that allow the vehicle to receive measured or anticipated solar energy. The measured or anticipated solar energy can be based on receiving threshold levels of light energy, including attempting to receive a maximum available light energy, a desired amount of light energy or limiting received light energy.

FIG. 8 illustrates an example operation of an autonomous driving system (not shown in FIG. 8) of a vehicle 800 caused by a sanitation system, such as the sanitation system 200 of FIG. 2, for elevating air temperature within the vehicle 800 by increasing heating based on external source, such as a solar load or external light/heat source. For example, the sanitation system 200 may be integrated as a part of the vehicle 800. More specifically, FIG. 8 illustrates one embodiment for orienting the vehicle 800 towards an external load (e.g., the sun) to maximize the heat source (e.g., sunlight entry) in the cabin 803 through windshield glass 801. For example, the vehicle 800 may be oriented by choosing a parking space exposed to sunlight or choosing a road lane exposed to sunlight, or other light source or heat source. The example operation of orienting the vehicle 800 towards the sun can be initiated by the sanitation system 200. For example, the processor(s) 202-1 may first determine no sensitive entities (such as perishable goods or medical substance or animals) are present in the cabin 803 of the vehicle 800 based on image sensor data generated by the plurality of sensors 210. The processor(s) 202-1 may also determine that external environment conditions (i.e., environment outside the vehicle 800) indicate sufficient or a threshold amount of sunlight based on light sensor data generated by the plurality of sensors 210. Alternatively, the processor(s) 202-1 may determine there is sufficient sunlight based on weather forecast data received from a wireless networking interface (not shown in FIG. 8) of the vehicle 800. The processor(s) 202-1 may further determine, based on light sensor data indicating direction of the sunlight and current vehicle orientation provided by an autonomous driving system of the vehicle 800, that the position and/or orientation of the vehicle 800 can be adjusted in a particular manner to result in more exposure of sunlight to the cabin 803 of the vehicle 800. Then, the processor(s) 202-1 may cause the autonomous driving system to re-position and/or re-orient the vehicle as illustrated in FIG. 8 to allow more exposure of sunlight into the cabin 803 of the vehicle 800. Although not illustrated in FIG. 8, in other embodiments, a sanitation system, such as the sanitation system 200, may cause the vehicle 800 to tilt itself by raising or lowering suspension elements of the vehicle 800 to facilitate direct sunlight entry into the cabin 803 of the vehicle 800. In still other embodiments, the sanitation system 200 may cause a door window or top window of the vehicle 800 to open to facilitate sunlight entry into the cabin 803 of the vehicle 800. In some other embodiments, the sanitation system 200 may cause the autonomous driving system of the vehicle 800 to drive to vehicle 800 to a nearby position that is less shaded based on sensor data generated by the plurality of sensors 210 to increase exposure to sunlight.

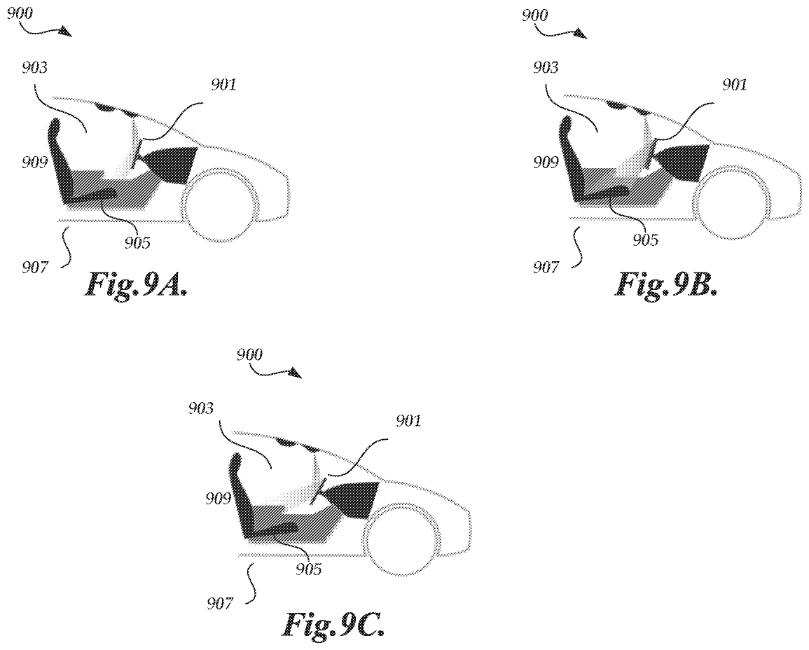

FIGS. 9A-9C illustrate example operations of a center display 901 of a vehicle 900 that can be caused by a sanitation system, such as the sanitation system 200 of FIG. 2, for reflecting sanitation lighting towards different surfaces in a vehicle cabin 903. More specifically, the center display 901 is tilted in different angles to reflect sanitation lighting towards specific surfaces inside the vehicle cabin 903. The sanitation lighting may be part of the light sources 206-1 in FIG. 2. As shown in FIG. 9A, the center display 901 is titled toward a rectangular degree with respect to a floor plane 907 of the vehicle 900. Such tilting results in the disinfection light be more focused on surface area in front of the seat 905. Such tilting may result from that the processor(s) 202-1 determines the surface area in front of the seat 905 has been touched more based on sensor data generated by the plurality of sensors 210. As such, the processor(s) 202-1 may cause a mechatronic component of the center display 901 to tilt in the angle as shown in FIG. 9A. In contrasts, FIGS. 9B and 9C show that the center display 901 be titled toward degrees such that the surface of the center display 901 is more horizontal to the floor plane 907 of the vehicle 900 compared with the tilting in FIG. 9A. As shown in FIG. 9B, the tilt enables more disinfection light to be directed toward the seat 905 of the vehicle 900. As shown in FIG. 9C, the tilt allows the disinfection light to be emitted toward the seatback 909 of the seat 905. Other specific touch surfaces of the vehicle, such as the cupholders, center console armrest, and seat cushion (illustrated in FIG. 3) may also receive focused disinfection light illumination from the sanitation lighting based on different tilting of the center display 901.

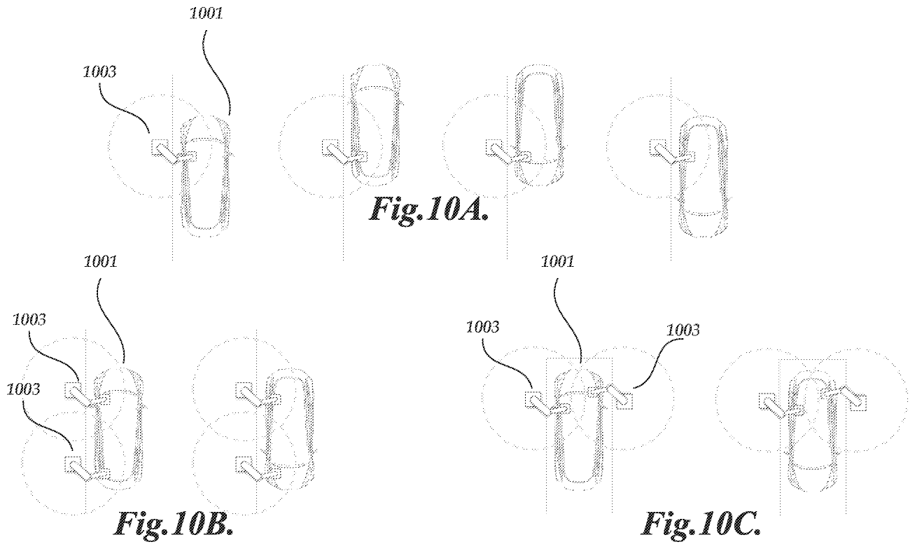

FIGS. 10A-10C illustrate example operations of service machinery performed on a vehicle 1001 that can be caused by a sanitation system, such as the sanitation system 200 of FIG. 2, for sanitizing the vehicle 1001. More specifically, the sanitation system 200 may work with one or more service robots 1003 to sanitize the interior of the vehicle 1001. FIG. 10A illustrates example operations showing the sanitation system 200 causes one service robot 1003 to sanitize the interior of the vehicle 1001 under different orientations of the vehicle 1001. The service robot 1003 may utilize contact-less infrared lamp heating, contact heating or steam for sanitizing the interior of the vehicle 1001. In some embodiments, upon determining the absence of sensitive entities within the cabin of the vehicle 1001 based on sensor data generated by the plurality of sensors 210, the processor(s) 202-1 may trigger the autonomous driving system of the vehicle 1001 to drive the vehicle 1001 to parking lots or service stations where the service robot 1003 is located. As shown in FIG. 10A, the service robot 1003 may be static in position and the processor(s) 202-1 may cause the autonomous driving system to adjust the orientation or the position of the vehicle 1001 with respect to the service robot 1003 to allow sanitation toward different area of the interior of the vehicle 1001. Additionally, the processor(s) 202-1 and the service robot 1003 may determine a protocol specifying steps both sides have to perform during the sanitation operations. For example, the processor(s) 202-1 may first de-activate the security alarm of the vehicle 1001 before the service robot 1003 may approach or contact the vehicle 1001. The processor(s) 202-1 may then cause the doors windows of the vehicle 1001 be lowered or opened, followed by the service robot 1003 emitting steam into the cabin of the vehicle 1001. The protocol may further specify that the processor(s) 202-1 will then cause the doors windows to be closed and the security alarm activated once the service robot 1003 has finished sanitizing the vehicle 1001.

FIG. 10B-10C illustrate example operations showing a sanitation system, such as the sanitation system 200, causes two service robots 1003 to sanitize the interior of the vehicle 1001 under different orientation of the vehicle 1001. In FIG. 10B, the two service robots 1003 are on the same side of the vehicle 1001 and the processor(s) 202-1 may cause the autonomous driving system of the vehicle 1001 to orient the vehicle 1001 in two different directions to allow each of the service robots 1003 sanitizing different parts of the interior of the vehicle 1001. In FIG. 10C, the two service robots 1003 are on the different sides of the vehicle 1001 and the processor(s) 202-1 may also cause the autonomous driving system of the vehicle 1001 to orient the vehicle 1001 in two different directions to allow each of the service robots 1003 sanitizing different parts of the interior of the vehicle 1001. In some embodiments, the service robots 1003 may sanitize the interior of the vehicle 1001 using ultraviolet light or microwaves. In other embodiments, the ultraviolet light source or microwaves source may be built into a component (such as the light sources 206-1 of FIG. 2) within the vehicle 1001.

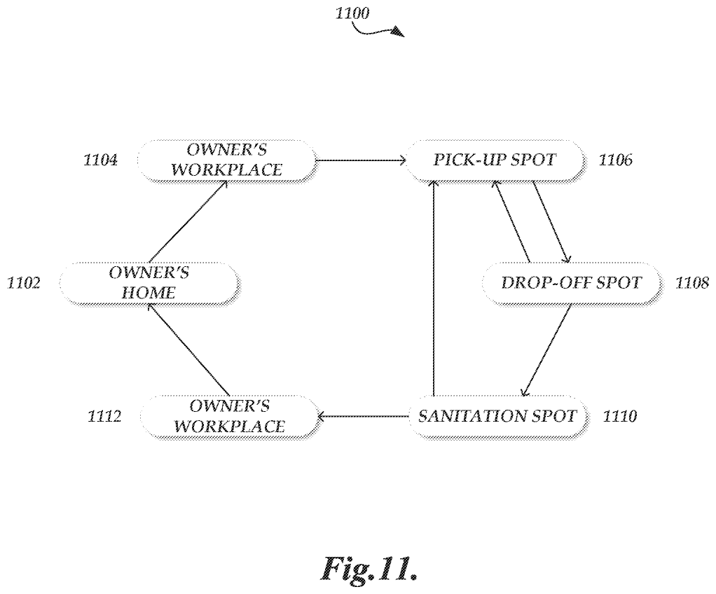

FIG. 11 illustrates an example rideshare cycle 1100 associated with a vehicle in which embodiments of the present disclosure can be implemented. More specifically, a sanitation system such as the sanitation system 200 can coordinate with components (e.g., an autonomous driving system) of a vehicle to sanitize the vehicle at proper timing. The rideshare cycle 1100 can begin at block 1102, where the vehicle may be at the owner's residence. The owner may then start riding the vehicle to the owner's workplace, during which the sensors 210 may detect the environmental conditions (e.g., whether the owner sneezes, the presence of certain pathogens, the temperature or humidity of the vehicle) associated with the vehicle. In some examples, the sanitation system 200 may not cause any sanitation routine to be implemented to sanitize the vehicle when the owner is riding the vehicle to his/her workplace.

At block 1104, the vehicle arrives at the workplace and the owner exits the vehicle. An autonomous driving system may then be provided with a pick-up spot where passengers other than the owner would ride with the vehicle. More specifically, the pick-up spot may be communicated to a network module of the vehicle and the network module may provide the pick-up spot to the autonomous driving system, which may then estimate a travel time from the owner's workplace to the pick-up spot. The sanitation system 200 may then decide whether to perform any sanitation operation on the vehicle based on the sensor data collected between the ride from owner's residence to the owner's workplace. For example, if the sensors 210 detected the presence of pathogens at particular touch surfaces after the occupant of the owner, the processor(s) 202-1 can generate a sanitation routine that causes the humidity equipment 208-1 to emit disinfection vapor toward the particular touch surfaces. In some examples, the sanitation routine generated by the processor(s) 202-1 may also depend on the estimated travel time from the owner's workplace to the pick-up spot. For example, if the estimated travel time is longer than thirty minutes, the processor(s) 202-1 may generate a sanitation routine that may cause the HVAC system 204 of the vehicle to elevate the temperature to a certain degree for thirty minutes to eliminate certain viruses. If the estimated travel time is shorter, the processor(s) 202-1 may generate a sanitation routine that takes less time to be completed. For example, the processor(s) 202-1 may cause the light sources 206-1 to emit disinfection light toward particular touch surfaces where pathogens are detected but not cause the HVAC system 204 to elevate temperature within the vehicle.

In some examples, a sanitation routine that employs only disinfection lighting for sanitation can be completed between two to twenty minutes. In some examples, a sanitation routine that utilizes the HVAC system 204 of the vehicle to increase the temperature and/or humidity for sanitation can take twenty minutes up to an hour. The sanitation system 200 can take the estimated travel time for picking up the next passenger along with the time required to achieve effective sanitation under different sanitation mechanisms into consideration to generate a sanitation routine that can be completed under the timing constraints imposed by the rideshare schedule. Additionally, a sanitation routine may be generated by the sanitation system 200 further based on the pathogens that are targeted to be eliminated. For example, if the time until the next passenger is picked up is more than thirty minutes and the sensor data generated by the sensors 210 indicate the presence of SARS-COV-2, the sanitation system 200 may generate a sanitation routine that triggers the HVAC system 204 of the vehicle to increase the temperature within the vehicle to around 56° C. for thirty minutes. If the time until the next pick up is less than thirty minutes, the sanitation system 200 may generate a sanitation routine that combines increasing the temperature within the vehicle to around 56° C. and spraying disinfection vapor along with disinfection lighting toward the touch surfaces where the presence of SARS-COV-2 is indicated according to the sensor data.

At block 1106, the vehicle arrives at the pick-up spot and picks up the non-owner passenger. The sanitation system 200 may keep monitoring the environmental conditions after the passenger is picked up. For example, the sensors 210 may keep detect the presence of pathogens during the occupancy of the passenger. In some examples, the sanitation system 200 may not cause any sanitation operation to be performed to sanitize the vehicle while the passenger is riding with the vehicle. At block 1108, the vehicle arrives at the drop-off spot where the passenger exits the vehicle. If there is another passenger waiting to be picked up in another pick-up spot, the rideshare cycle proceeds back to block 1106, where another passenger is picked-up to ride with the vehicle. During the travel from block 1108 to block 1106, the sanitation system 200 may cause a component of the vehicle to implement some sanitation operations, which may be dependent upon the estimated travel time from block 1108 back to block 1106 and the environmental conditions detected by the sensors 210. For example, if the travel time is above fifteen minutes, the sanitation system 200 may cause the humidity equipment 208-1 to spray disinfection vapor and then cause the HVAC system 204 to remove the vapor before the next passenger is picked-up. If the estimated travel time is below five minutes, the sanitation system 200 may only sanitize the vehicle using disinfection lighting. In some embodiments, the rideshare cycle may iteratively proceed back and forth between block 1106 and 1108 until there is no next passenger already waiting to rideshare the vehicle.

In some embodiments, if there is no next passenger waiting to rideshare the vehicle, the vehicle may proceed to block 1110 where the vehicle arrives at a sanitation spot where more sanitation operations can be performed on the vehicle. In some embodiments, the sanitation spot may be the same spot where the last ridesharing passenger is dropped off. In some embodiments, may be a roadside or a parking lot nearby. For example, after dropping of the last rideshare passenger at block 1108, the vehicle may proceed to block 1110 (e.g., a roadside or a parking lot) where a position and orientation of the vehicle allows more exposure of the sunlight into the vehicle, such as the scenario illustrated in FIG. 8. In some embodiments, the block 1110 may be a charging station where service machinery is stationed and the vehicle may coordinate with service machinery to sanitize the vehicle, such as the scenario illustrated in FIGS. 10A-10C. In some embodiments, at block 1110, the processor(s) 202-1 may cause a HVAC system 204 of the vehicle to increase the temperature and humidity of the vehicle and maintain the increased temperature and humidity for periods of time (e.g., thirty minutes) that might be effective in reducing some types of pathogens. After the more thorough sanitation operations (compared with the sanitation operations performed between block 1106 and block 1108) performed, the rideshare cycle 1100 may proceed back to block 1106, where the vehicle arrives at another pick-up spot to pick up a next passenger. The rideshare cycle 1100 may then repeat the previously described operations associated with blocks 1106, 1108 and 1110.

Alternatively, the rideshare cycle 1100 would proceed to block 1112 to pick-up the owner of the vehicle. For example, when the owner is about to leave the owner's workplace and gets back to the owner's residence, the rideshare cycle 1100 may proceed from block 1110 to block 1112 to pick-up the owner at the owner's workplace. Advantageously, the vehicle is well-sanitized while at block 1110, and the owner may ride the vehicle from workplace to home under a well-sanitized environment.

In some examples, the sanitation system 200 may implement different sanitation mechanisms (e.g., emitting disinfection lighting, emitting disinfection vapor, or adjusting temperature) or adjust the frequency of sanitation operations based on information associated with the rideshare cycle 1100. In some embodiments, the sanitation routine that the sanitation system 200 generates may be based on locations of the pick-up spots. For example, if the vehicle 100 just picked up a passenger from a location (e.g., a hospital, a crowded venue or the like) where the passenger was likely to be exposed to pathogens, viruses or bacteria, the sanitation system 200 may cause a sanitation routine to be implemented immediately after that passenger is dropped off. The sanitation routine implemented may be operations such as emitting disinfection lighting or vapor that can be completed within shorter period of time if the next passenger is to be picked up within five to ten minutes. Alternatively, the sanitation system 200 may communicate with the autonomous driving system of the vehicle 100 or the ride-sharing application used by the next passenger to delay the pickup such that the needed sanitation operations (e.g., elevating the temperature within the vehicle to certain temperature for over twenty minutes or half an hour) can be completed.

In some examples, the frequency or types of sanitation routines implemented can be adjusted based on seasons (e.g., winter or summer), weather (e.g., rainy or sunny), geographic locations (e.g., urban or rural areas), and/or other publicly available information (e.g., pandemic alert in certain area issued by health authorities). For example, during the winter when flu is more widely spread, the sanitation system 200 may increase the frequency of sanitizing the vehicle 100. As another example, during the dry and sunny days of summer, the sanitation system 200 may generate a sanitation routine that includes rolling down the windows of the vehicle 100 to allow exposure to sunlight and spraying disinfection vapor within the cabin of the vehicle 100 but does not include increase the temperature using the HVAC system 204 of the vehicle 100.

Additionally and optionally, the sanitation system 200 may define different levels of sanitized conditions within the vehicle 100. Based on the sanitation operations that have been performed on the vehicle 100 and/or sensor data generated by the sensors 210 that indicate the amount of presence of pathogens, bacteria or viruses within the vehicle 100, the sanitation system 200 may define the sanitized conditions of the vehicle 100 on a relative scale, such as 25% sanitized, 50% sanitized, 75% sanitized or fully-sanitized. The sanitation system 200 may coordinate with other components (e.g., a Wi-Fi or other wireless communication modules) of the vehicle 100 to transmit the sanitized conditions to the ride-sharing applications of passengers that may be potentially picked up by the vehicle 100. As such, the passengers may be aware of the sanitized conditions of the vehicles they might ride and make more informed decision on whether to ride with the vehicles or put on protective gear (e.g., mouth mask) before riding with the vehicles. In some examples, the sanitation system 200 may receive preferences of sanitized conditions from the passenger that will be served next and implement a sanitation routine that can help the vehicle to reach the level of sanitation preferred or requested by the passenger. For instance, the next passenger may request that the vehicle 100 be 100% sanitized, which can be communicated to the sanitation system 200 through a wireless communication module of the vehicle 100. The sanitation system 200 may then implement sanitation operations including heating, disinfection lighting and spraying disinfection vapor to bring the vehicle 100 to the highest attainable sanitized condition. Additionally, the sanitation system 200 may cause the autonomous driving system of the vehicle 100 to drive the vehicle 100 to a spot where service machinery is located to sanitize the vehicle 100 in ways depicted in FIGS. 10A-10C to reach 100% sanitized condition if all the sanitation operations the vehicle 100 itself is capable of performing cannot result in 100% sanitized condition.

In still other embodiments, the sanitation system 200 may coordinate with components of the vehicle 100 to convey sanitation conditions associated with the vehicle 100 to a passenger. In some embodiments, the sanitation system 200 may determine that certain area within the vehicle 100 is under-sanitized based on sensor data generated by the sensors 210, and the sanitation system 200 may then notify a passenger not to be exposed to the under-sanitized area. For example, the sensors 210 may detect that some pathogens are present around the front passenger seat. In response, the sanitation system 200 may cause a user interface (e.g., a center display) of the vehicle 100 to alert the passenger not to sit on the front passenger seat.

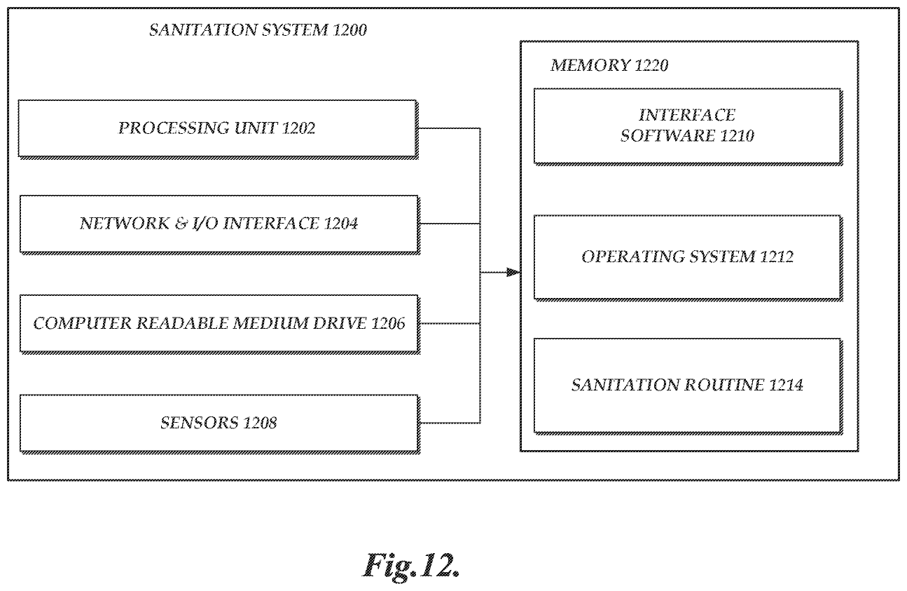

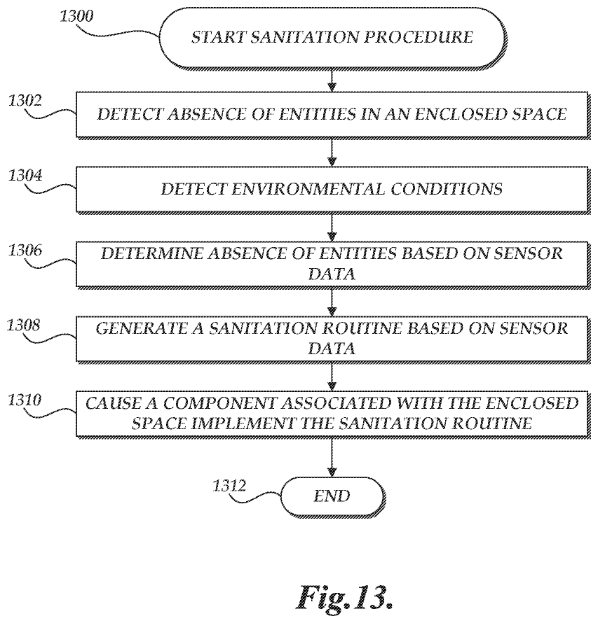

With reference now to FIG. 12, an illustrative architecture of a sanitation system 1200 for sanitizing a vehicle will be described. The sanitation system 200 described above may adopt the same or similar architecture as described in FIG. 12. The sanitation system 1200 may perform the same or similar operations performed by the sanitation system 200. The sanitation system 1200 may coordinate with components of a vehicle to sanitize the vehicle, where the components of the vehicle may include, but not limited to, the HVAC system 204, the lighting controls 206 and the humidity controls 208 as described in FIG. 2. At least some parts of the sanitation system 1200 may be parts of components/systems of a vehicle. For example, some of the sensors 1208 may be a part of an autonomous driving system of the vehicle. Alternatively, the sanitation system 1200 may be a stand-alone module that interacts with other components of a vehicle.

The architecture of FIG. 12 is illustrative in nature and should not be construed as requiring any specific hardware or software configuration for the sanitation system 1200. The general architecture of the sanitation system 1200 depicted in FIG. 12 includes an arrangement of computer hardware and software components that may be used to implement aspects of the present disclosure. As illustrated, the sanitation system 1200 includes a processing unit 1202, a network and I/O (input/output) interface 1204, a computer readable medium drive 1206, and sensors 1208, all of which may communicate with one another by way of a communication bus (not explicitly shown in FIG. 12). Some of the components of the sanitation system 1200 may be physical hardware components or implemented in a virtualized environment. For example, the memory 1220 may be a virtualized memory that is hosted by a cloud provider network that is remote to a vehicle where other parts of the sanitation system 1200 are deployed.

The network and I/O interface 1204 may provide connectivity to one or more networks or computing systems, such as providing communication to the service machinery illustrated in FIG. 10A-10C. Additionally, the network and I/O interface 1204 may communicate with other networks to obtain environmental conditions (e.g., weather forecast or data) that can be utilized by the processing unit 1202 to determine what sanitation operations are to be performed. The processing unit 1202 may thus receive information and instructions from other computing systems or services via the network and I/O interface 1204. The processing unit 1202 may also communicate to and from memory 1220 and further provide output information to the network and I/O interface 1204. For example, the network and I/O interface 1204 may function as a user interface through which the processing unit 1202 may obtain answers indicating environmental conditions of a vehicle (e.g., whether heat-sensitive entities such as medicine, electronic equipment or humans are present) from a user. In some embodiments, the processing unit 1202 may determine the absence of sensitive entities within an enclosed space based on sensor data collected by the sensors 1208. In some embodiments, the sanitation system 1200 may include more (or fewer) components than those shown in FIG. 12.

The memory 1220 may include computer program instructions that the processing unit 1202 executes in order to implement one or more embodiments in accordance with the present disclosure. The memory 1220 may include RAM, ROM, or other persistent or non-transitory memory. The memory 1220 may store an operating system 1212 that provides computer program instructions for use by the processing unit 1202 in the general administration and operation of the sanitation system 1200. The memory 1220 may further include the interface software 1210 for transmitting and receiving computer program instructions and other information for implementing aspects of the present disclosure. For example, in one embodiment, the memory 1220 stores a sanitation routine 1214 that defines one or more sanitation operations to be performed. More specifically, the sanitation routine 1214 may define the operations illustrated in FIGS. 9A-9C where sanitation lighting is emitted with a center display of a vehicle titled gradually in different angles for reflecting sanitation lighting towards different surfaces inside a vehicle cabin. Alternatively, the sanitation routine 1214 may define the operations illustrated in FIGS. 4A-4C, FIG. 5, FIGS. 6A-6B, FIGS. 7A-7C, FIG. 8 and/or FIGS. 10A-10C. For example, the sanitation routine 1214 may define that the seatbelt 601 be rolled out from the configurations as illustrated in FIG. 6A to FIG. 6B. Based on the operations defined in the sanitation routine 1214, the processing unit 1202 may cause corresponding component(s) associated with an enclosed space to implement the sanitation routine 1214. For example, when the sanitation routine 1214 defines that a vehicle be oriented towards the sun to increase sunlight entry into a vehicle cabin as illustrated in FIG. 8, the processing unit 1202 may cause an autonomous driving system of the vehicle to implement the sanitation routine 1214 to orient the vehicle towards the sun. In some embodiments, the sanitation routine 1214 may be generated based on sensor data collected by the sensors 1208. For example, when the sensor data indicates there is sufficient sunlight from the west, the processing unit 1202 may generate the sanitation routine 1214 that defines an operation of orienting a vehicle toward the west. The processing unit 1202 may then cause an autonomous driving system of the vehicle to implement the sanitation routine 1214 to orient the vehicle toward the west (assuming the vehicle is not currently oriented toward the west). In such a scenario, the processing unit 1202 may not generate the sanitation routine 1214 that defines an operation of orienting the vehicle toward the east. In some embodiments, the processing unit 1202 generates the sanitation routine 1214 in response to determining that there are no sensitive entities present in an enclosed space based on sensor data collected by the sensors 1208.

Turning now to FIG. 13, an illustrative sanitation procedure 1300 for sanitizing an enclosed space, such as the enclosed space 110 of the vehicle 100, will be described. The sanitation procedure 1300 may be implemented, for example, by the sanitation system 200 of FIG. 2 or the sanitation system 1200 of FIG. 12.

The sanitation procedure 1300 begins at block 1302, where the sanitation system 200 or the sanitation system 1200 may detect absence of entities in the enclosed space. More specifically, the sensors 1208 may detect absence of entities in the enclosed space and generate first sensor data that might indicate absence of the entities. The first sensor data may be images captured by the sensors 1208 that, when analyzed by the processing unit 1202, directly reflect the occupancy of the enclosed space. Alternatively, the first sensor data may include information that can be analyzed by the processing unit 1202 to indirectly infer the presence or absence of entities in the enclosed space. For example, the first sensor data may include history of the opening and closing of a door of the enclosed space. The first sensor data may also indicate the presence of other sensitive entities such as pets, medicine or electronic devices. The sensors 1208 that are used to generate the first sensor data may include vision sensor(s) (e.g., for analyzing images from interior or exterior of an enclosed space), acoustic sensor(s), thermal sensor(s), weight sensor(s), pressure sensor(s), capacitive sensor(s), radio frequency sensor(s) (e.g., for detecting human body movement, breathing, or heartbeat), laser sensor(s), or gas detectors.

At block 1304, the sanitation system 200 or the sanitation system 1200 detects environmental conditions associated with the enclosed space. In one example, some of the sensors 1208 may detect environmental conditions associated with the enclosed space to generate second sensor data that is indicative of the environmental conditions, which may include temperature and/or humidity of the enclosed space. Additionally, the environmental conditions may include the presence of certain particulates or pathogens in the air of the enclosed space or on touch surfaces of the enclosed space. For example, some of the sensors 1208 may utilize illumination to detect contamination or cleanliness of touch surfaces. The detection may include illuminating touch surfaces with light and imaging the touch surfaces to detect bacteria, viruses, pathogens and/or dirt. Further, the illumination may be adjusted to selectively target specific touch surfaces where more touch or contact events might occur. Besides detecting conditions within the enclosed space, some of the sensors 1208 can detect environmental conditions external to the enclosed space, such as whether there is sunlight or whether it is raining outside the enclosed space. For example, some of the sensors 1208 may include cameras that capture images of the environment external to the enclosed space or may include light sensors for sensing which direction the sunlight comes from.

Based on the sensor data gathered at block 1302, at block 1306, the sanitation system 200 or 1200 may determine absence of sensitive entities within an enclosed space. More specifically, the processing unit 1202 may determine the absence of sensitive entities within the enclosed space based on the first sensor data that is indicative of the absence of sensitive entities. For example, the first sensor data may include images captured by some of the sensors 1208 and the processing unit 1202 may analyze these images to determine whether the enclosed space is occupied by any sensitive entities or not. Image processing and machine learning techniques may be employed to determine the absence or presence of sensitive entities such as humans, medicine, pets and/or electronic devices. Additionally, the first sensor data may include acoustic signals (e.g., human speech) captured by some of the sensors 1208 and the processing unit 1202 may analyze these acoustic signals to determine the presence of humans or pets. Alternatively, and additionally, the first sensor data may include history of opening and closing of a door of the enclosed space collected by capacitive touch sensors and the processing unit 1202 may utilize this history to infer the occupancy of the enclosed space. Advantageously, using sensor data gather by different types of sensors may increase the accuracy of determining the presence of sensitive entities within the enclosed space.

At block 1308, the sanitation system 1200 may generate a sanitation routine 1214 based on sensor data collected by the sensors 1208. In some embodiments, the processing unit 1202 generates the sanitation routine 1214 in response to determining that no sensitive entities are within the enclosed space, as performed in block 1306. The processing unit 1202 may utilize the first sensor data collected at block 1306 and/or second sensor data collected at block 1304 to generate the sanitation routine 1214. For example, the sensor data may include images captured by an infrared (IR) camera that indicates the enclosed space was once occupied by an occupant having a fever. The processing unit may then utilize information provided by the IR camera to generate the sanitation routine 1214 that may include emitting disinfection light (e.g., by the light sources 206-1) directed toward the area that was once occupied by the occupant. As another example, the sensor data may include acoustic signals that might be indicative of audible sickness indicators such as coughing, sneezing, sniffing or blowing noise of a current or past occupant of the enclosed space. The processing unit 1202 may then analyze the acoustic signals to generate the sanitation routine 1214 that may include spraying disinfection vapor (e.g., by the humidity equipment 208-1) to sanitize the air within the enclosed space. As still another example, the sensor data may indicate that some of the seats or surfaces were contacted/touched by occupants. As such, the processing unit 1202 may analyze these sensor data to generate the sanitation routine 1214 that may be specifically directed to those seats or surfaces that were contacted/touched. For example, the sanitation routine 1214 may include rolling out a seatbelt of a particular seat that was occupied and emit disinfection light onto the seatbelt, as illustrated in FIGS. 6A and 6B. The sanitation routine 1214 may further include recline forward or backward a back of the seat as illustrated in FIG. 5 for disinfection light to be applied different surfaces of the seat.

At block 1310, the sanitation system 1200 may cause a component associated with the enclosed space to implement the sanitation routine 1214. The component may be the HVAC system 204, the lighting controls 206 and/or humidity controls 208 as illustrated in FIG. 2. Additionally, the component may be an autonomous driving system of a vehicle. For example, the sanitation routine 1214 may be positioning and orienting the vehicle in a particular manner such that sunlight can penetrate into the interior of the vehicle through a windshield, such as the scenario illustrated in FIG. 8. The processing unit 1202 may then cause the autonomous driving system of the vehicle to position and orient the vehicle in the desired position and orientation to allow exposure of sunlight. In other examples, the sanitation routine 1214 may be adjusting the temperature and humidity within the enclosed space to a particular range that inhibits certain pathogens or viruses (e.g., reducing half-life of SARS-CoV-2 as discussed above). As such, the processing unit 1202 may then cause the HVAC system 204 and/or a component (e.g., the heating element and/or passive material illustrated in FIGS. 7C-7A) to adjust the temperature and/or humidity within the enclosed space. For example, when current humidity within the enclosed space detected by the sensors 1208 are 50% and current temperature within the enclosed space detected by the sensors 1208 are 65° F., the processing unit 1202 may generate the sanitation routine 1214 that specifies a target humidity of 80% and temperature of 75° F. for reducing the half-life of the SARS-COV-2. The processing unit 1202 may then cause the humidity equipment 208-1 and the HVAC system 204 to implement the sanitation routine 1214 to increase the humidity from 50% to 80% and increase temperature from 65° F. to 75° F. Advantageously, the automatic detection of environmental conditions associated with the enclosed space to automatically implement the sanitation routine 1214 that may be specifically tailored to the detected environmental conditions would avoid the time-consuming and laborious manual sanitation. Further, compared with manual sanitation, the sanitation procedure 1300 may better avoid the situations where an enclosed space is not sanitized frequently enough.

After implementing the aforementioned blocks, thus sanitizing an enclosed space, the sanitation procedure 1300 ends at block 1312. If the sanitation procedure 1300 is implemented by the sanitation system 200 of FIG. 2 or the sanitation system 1200 of FIG. 12 to sanitize the enclosed space 110 of the vehicle 100 of FIG. 1, the vehicle 100 will be in a sanitized condition that can help prevent the spread of communicable diseases when different persons ride with the vehicle 100.

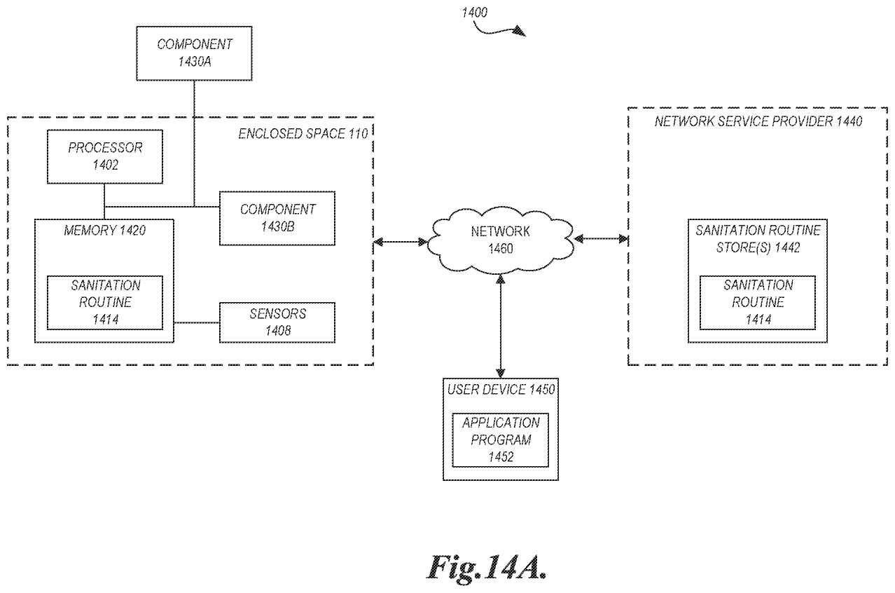

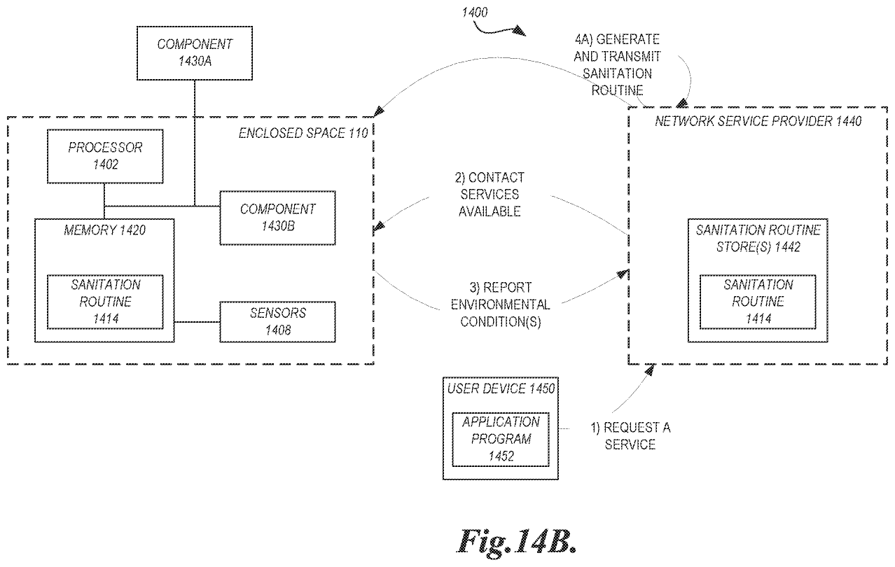

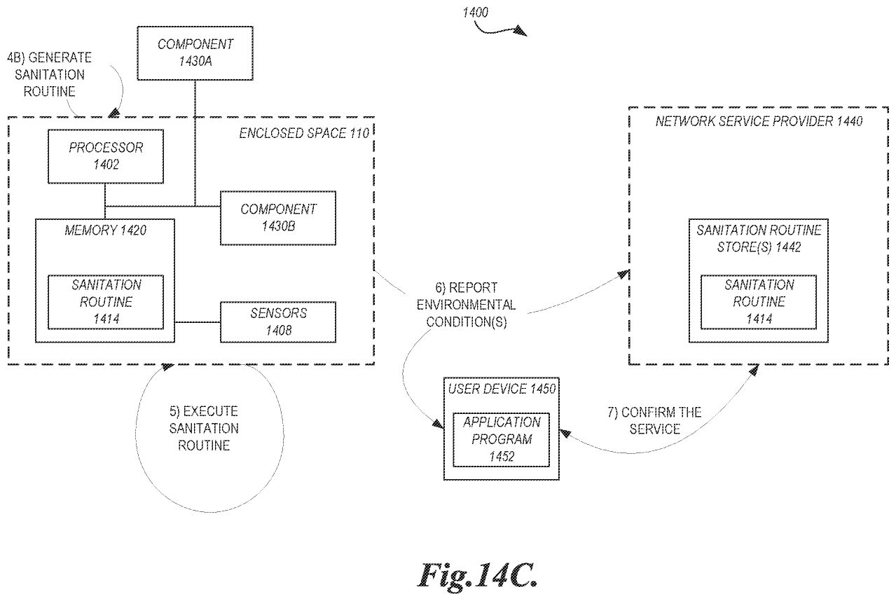

FIG. 14A illustrates an environmental condition management system 1400 in which an enclosed space 110 (e.g., a vehicle cabin or a building room) can be sanitized to provide services to multiple users in accordance with one or more aspects of the present application. The environmental condition management system 1400 includes a collection of local resources to the enclosed space 110 that may be utilized to manage environmental condition of the enclosed space 110, such as a cabin of the electric vehicle 100. The collection of local resources may include a processor 1402, a memory 1420, one or more sensors 1408, and one or more components 1430A (e.g., a service robot outside the enclosed space 110) and 1430B (e.g., a HVAC system within the enclosed space 110) that can be utilized to adjust environmental condition of the enclosed space 110. The enclosed space 110 may include a processor 1402 for obtaining (e.g., from the network service provider 1440) or generating a sanitation routine 1414 that, when executed by the processor 1402, causes the components 1430A or 1430B to adjust a current environment of the enclosed space 110. More specifically, the sanitation routine 1414 may be generated by the processor 1402 based at least in part on sensor data generated by the sensors 1408, which detect a current environmental condition of the enclosed space 110. The current environmental condition of the enclosed space 110 may include at least one of a temperature, a humidity, presence of one or more pathogens, and user sharing history or any combinations thereof. For example, the sensors 1408 may detect that the current temperature of the enclosed space 110 is 58° F., the current humidity of the enclosed space 110 is 60%, there is presence of SARS-COV-2 around the front seat of the enclosed space 110, and/or that a user who has a body temperature indicative of fever just occupied the enclosed space 110.

The sanitation routine 1414 may specify a target environmental condition that is desired for the enclosed space 110. The sanitation routine 1414 may further specify one or more particular components associated with the enclosed space 110 (e.g., the components 1430A and/or 1430B) that will be utilized to adjust the current environmental condition of the enclosed space 110 toward the target environmental condition. For example, the sanitation routine 1414 may specify that a target environmental condition of the enclosed space 110 is 75° F. and 80% and that a HVAC system of the enclosed space 110 will be utilized to adjust the temperature and humidity within the enclosed space 110 toward 75° F. and 80%, respectively.

The local resources to the enclosed space 110 are represented in a simplified, logical form and do not reflect all of the physical software and hardware components that may be implemented to provide the functionality associated with the local resources.

Alternatively, the sanitation routine 1414 may also be obtained by the processor 1402 from the network service provider 1440 through the network 1460. As shown in FIG. 14A, the environmental condition management system 1400 further includes the network service provider 1440 that can communicate with one or more of the local resources associated with the enclosed space 110 via computer network connections provided by the network 1460. As such, the network service provider 1440 may assist managing the environmental condition(s) of the enclosed space 110 remotely. The network 1460 may be any wired network, wireless network, or combination thereof. In addition, the network 1460 may be a personal area network, local area network, wide area network, cable network, fiber network, satellite network, cellular telephone network, data network, or combination thereof. In the example environment of FIG. 14A, the network 1460 is a global area network (GAN), such as the Internet. Protocols and components for communicating via the other aforementioned types of communication networks are well known to those skilled in the art of computer communications and thus, need not be described in more detail herein. The network service provider 1440 is represented in a simplified, logical form and does not reflect all of the physical software and hardware components that may be implemented to provide the functionality associated with the network-based service(s).

In some embodiments, the network service provider 1440 can generate the sanitation routine 1414 that, when obtained and executed by the processor 1402, causes operations on the part of the components 1430A-1430B similarly to the scenario when the sanitation routine 1414 is generated by the processor 1402. The network service provider 1440 may generate the sanitation routine 1414 based on a request received from a user device 1450. The user(s) operating the user device 1450 can be an individual customer or a corporate client of the network service provider 1440. The request may specify the demanded level of sanitation conditions of the enclosed space 110, where the level of sanitation conditions can be defined in detail by the network service provider 1440 and be made known to users in advance. The user may utilize the application program 1452 provided by the network service provider 1440 to request services of using the enclosed space 110 and communicate with the network service provider 1440. Additionally, or alternatively, the network service provider 1440 can generate the sanitation routine 1414 based on current environmental condition of the enclosed space 110, where the current environmental condition can be detected by the sensors 1408 and transmitted to the network service provider 1440 through the network 1460. In some embodiments, the network service provider 1440 can generate in advance different sanitation routines in the sanitation routine store 1442, where each of the different sanitation routines may specify different target environmental conditions to be achieved for the enclosed space 110 as well as different components associated with the enclosed space 110 that will be utilized to adjust environmental conditions of the enclosed space 110 toward a corresponding target environmental condition. Advantageously, the off-line generations of sanitation routine may shorten the total amount of time required to adjust the environmental condition of the enclosed space 110 to a desired sanitation condition.

FIG. 14B depicts illustrative interactions between elements of the environmental condition management system 1400 of FIG. 14A to adjust an environmental condition of the enclosed space 110. The interactions of FIG. 14B begin at (1), where the user device 1450 sends a request for using an enclosed space 110 through the application program 1452 to the network service provider 1440. In the context of rideshare, the request may specify the time and location the user would like to be picked up by a vehicle. The request may further specify the level of sanitation the user demands on the enclosed space 110 (e.g., the vehicle cabin) of the vehicle. In some embodiments, the identity of the user can be made immediately known to the network service provider 1440 when the user sends the request of service via the application program 1452. Then, the level of sanitation or target environmental condition demanded by the user can be inferred from the identity of the user. For example, if the request is sent by a corporate client, the network service provider 1440 can know that based on preferences indicated by the client previously the enclosed space 110 has to be sanitized to the highest standard in accordance with pre-defined protocol(s) agreed between the network service provider 1440 and the corporate client.

Thereafter, at (2), the network service provider 1440 contacts enclosed spaces (e.g., the enclosed space 110) that can potentially serve the user based on the request of service received in (1). In the context of rideshare, the network service provider 1440 would communicate with vehicles under its management that can likely meet the demands of the user based on time, location and sanitation level requested by the user. In some examples, the request in (1) may designate a particular vehicle for service and request a well-sanitized condition, and the network service provider 1440 will then manage to dispatch the vehicle to the user under the requested time, location and sanitation condition. As described previously, the network service provider 1440 can reach out to the vehicles through the network connections provided by the network 1460. While contacting the vehicle, the network service provider 1440 may provide information about the user (e.g., user location and requested pick-up time) to the processor 1402 of the enclosed space 110 of the vehicle.

At (3), in response to obtaining the request for service, the enclosed space 110 can report its current environmental condition to the network service provider 1440. As described above, the current environmental condition of the enclosed space 110 can be used by the network service provider 1440 to generate the sanitation routine 1414. The current environmental condition of the enclosed space 110 may be generated by the sensors 1408, stored in the memory 1420, and retrieved from the memory 1420 to be transmitted to the network service provider 1440. Along with the current environmental condition of the enclosed space 110, the location of the enclosed space 110 can also be transmitted to the network service provider 1440.

In response to receiving the environmental condition of the enclosed space 110, at (4A), the network service provider 1440 can generate the sanitation routine 1414 and transmit the sanitation routine 1414 to the enclosed space 110. The sanitation routine 1414 can be generated based at least in part upon the requested level of sanitation, the current environmental condition of the enclosed space, the time and location of the service. For example, if the user requests the highest level of sanitation, the current environmental condition shows the enclosed space 110 is far away from the requested sanitation condition and the user has to be picked up in a short amount of time, the network service provider 1440 will generate the sanitation routine 1414 that can meet these constraints. As an example, if the position of a vehicle reported in (3) shows that the vehicle is close to a service robot, the sanitation routine 1414 generated by the network service provider 1440 may, when executed by the processor 1402, cause the autonomous driving system of the vehicle to drive the vehicle to the place where the service robot is stationed for sanitation. The sanitation routine 1414 generated by the network service provider 1440 may further provide instructions to the service robot that requests the service robot to reach the highest level of sanitation (e.g., by combining disinfection lighting, heating, and vaporing) within certain amount (e.g., ten minutes) of time. As another example, the sanitation routine 1414 may provide that the HVAC system, the disinfection light sources, heating plates and vaporizers all be utilized concurrently to elevate the vehicle cabin to certain temperature and humidity for certain period of time (e.g., 56° C. and 70% for thirty minutes if the pick-up time is one hour away).

Alternatively, with reference now to (4B) in FIG. 14C, the sanitation routine 1414 can be generated by the enclosed space 110, more specifically, by the processor 1402 associated with the enclosed space 110 rather than generated and obtained from the network service provider 1440. The generation of the sanitation routine 1414 by the processor 1402 can be the same or similar to the generation of the sanitation routine 1414 by the network service provider 1440. For example, the processor 1402 can generate the sanitation routine 1414 based at least in part on the current environment of the enclosed space 110 and the requested sanitation condition of the user. Additionally, the sanitation routine 1414 may further be generated based on other factors, such as weather information, rideshare history (e.g., locations the vehicle has been traveled recently) or the like that may be available to the processor 1402. For example, when the weather is sunny and above certain degree of temperature and humidity, the sanitation routine 1414 may specify windows of the vehicle be rolled down to allow the vehicle cabin to be exposed to the heat and humidity outside the vehicle instead of turning on the HVAC system of the vehicle to provide additional heat and humidity to the vehicle cabin. In contrast, if the weather is cloudy and both the temperature and the humidity are low (e.g., below 58° F. and 30%) outside, the sanitation routine 1414 may provide the HVAC system to be turned on to increase the temperature and humidity to a target level (e.g., below 75° F. and 70%).

At (5), the sanitation routine 1414 is executed by the enclosed space 110. Specifically, the processor 1402 may execute the sanitation routine 1414 to adjust the environmental condition of the enclosed space 110. As indicated above, the sanitation routine 1414, when executed by the processor 1402, may cause a component associated with the enclosed space 110 to adjust the current environment of the enclosed space toward a target environmental condition. For example, the sanitation routine 1414 may specify a target environmental condition of temperature of 75° F. and humidity of 70%, and that the HVAC system within the enclosed space 110 of a vehicle will be utilized to adjust the current temperature and humidity of the enclosed space 110 to temperature of 75° F. and humidity of 70%. Alternatively, if the temperature outside the vehicle is above 75° F. and humidity outside the vehicle is above 70%, the sanitation routine 1414, when executed by the processor 1402, will cause side window(s) and/or top window(s) to roll open to adjust the temperature and humidity of the vehicle toward the target levels. Additionally, and optionally, before executing the sanitation routine 1414, the processor 1402 may determine whether any sensitive entities are present within the enclosed space 110. Such determination can be based on sensor data indicative of the presence of sensitive entities generated by the sensors 1408. In some embodiments, the processor 1402 executes the sanitation routine 1414 after determining that no sensitive entities are present within the enclosed space 110. Additionally, the sensors 1408 can monitor the environmental condition of the enclosed space during and/or after the execution of the sanitation routine 1414.

Thereafter, at (6), the enclosed space 110 may report to the network service provider 1440 and/or the user device 1450 the environmental condition of the enclosed space after the sanitation routine 1414 is executed. The report can include the environmental condition (e.g., whether there is presence of some pathogen) of the enclosed space 110 after the execution of the sanitation routine 1414. The report can further include the approaches (e.g., using disinfection lighting, vapor, heating, or employing service robot) that are adopted by the enclosed space 110 for sanitation. Additionally, the report can include certification information stating that the sanitation condition of the enclosed space has been certified to be in compliance with some public or proprietary sanitation standards and/or protocols. In some embodiments, the report can be displayed through the application program 1452 installed on the user device 1450 to allow the user to be informed of the sanitation condition of the enclosed space. In some embodiments, the report can include information indicating that some areas of the enclosed space 110 have not reached sanitation condition requested by the user or that the user is advised not to contact some areas (e.g., a front seat of a vehicle) of the enclosed space 110.

At (7), the request of the service may be confirmed between the user device 1450 and the network service provider 1440. For example, the user device 1450 may communicate to the network service provider 1440 through the application program 1452 that the user still intends to be served at the earlier requested time when the sanitation condition of the enclosed space 110 after the execution of the sanitation routine 1414 does not reach the level requested by the user device 1450. As another example, the user device 1450 may communicate to the network service provider 1440 through the application program 1452 that the user would like to wait to be served until the environmental condition of the vehicle has reached the sanitation level demanded by the user.

The foregoing disclosure is not intended to limit the present disclosure to the precise forms or particular fields of use disclosed. As such, it is contemplated that various alternate embodiments and/or modifications to the present disclosure, whether explicitly described or implied herein, are possible in light of the disclosure. Having thus described embodiments of the present disclosure, a person of ordinary skill in the art will recognize that changes may be made in form and detail without departing from the scope of the present disclosure. Thus, the present disclosure is limited only by the claims.

The processes described herein or illustrated in the figures of the present disclosure may begin in response to an event, such as on a predetermined or dynamically determined schedule, on demand when initiated by a user or system administrator, or in response to some other event. When such processes are initiated, a set of executable program instructions stored on one or more non-transitory computer-readable media (e.g., hard drive, flash memory, removable media, etc.) may be loaded into memory (e.g., RAM) of a server or other computing device. The executable instructions may then be executed by a hardware-based computer processor of the computing device. In some embodiments, such processes or portions thereof may be implemented on multiple computing devices and/or multiple processors, serially or in parallel.

Depending on the embodiment, certain acts, events, or functions of any of the processes or algorithms described herein can be performed in a different sequence, can be added, merged, or left out altogether (e.g., not all described operations or events are necessary for the practice of the algorithm). Moreover, in certain embodiments, operations or events can be performed concurrently, e.g., through multi-threaded processing, interrupt processing, or multiple processors or processor cores or on other parallel architectures, rather than sequentially.

The various illustrative logical blocks, modules, routines, and algorithm steps described in connection with the embodiments disclosed herein can be implemented as electronic hardware (e.g., ASICs or FPGA devices), computer software that runs on computer hardware, or combinations of both. Moreover, the various illustrative logical blocks and modules described in connection with the embodiments disclosed herein can be implemented or performed by a machine, such as a processor device, a digital signal processor (“DSP”), an application specific integrated circuit (“ASIC”), a field programmable gate array (“FPGA”) or other programmable logic device, discrete gate or transistor logic, discrete hardware components, or any combination thereof designed to perform the functions described herein. A processor device can be a microprocessor, but in the alternative, the processor device can be a controller, microcontroller, or state machine, combinations of the same, or the like. A processor device can include electrical circuitry configured to process computer-executable instructions. In another embodiment, a processor device includes an FPGA or other programmable device that performs logic operations without processing computer-executable instructions. A processor device can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor device may also include primarily analog components. For example, some or all of the rendering techniques described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of a method, process, routine, or algorithm described in connection with the embodiments disclosed herein can be embodied directly in hardware, in a software module executed by a processor device, or in a combination of the two. A software module can reside in RAM memory, flash memory, ROM memory, EPROM memory, EEPROM memory, registers, hard disk, a removable disk, a CD-ROM, or any other form of a non-transitory computer-readable storage medium. An exemplary storage medium can be coupled to the processor device such that the processor device can read information from, and write information to, the storage medium. In the alternative, the storage medium can be integral to the processor device. The processor device and the storage medium can reside in an ASIC. The ASIC can reside in a user terminal. In the alternative, the processor device and the storage medium can reside as discrete components in a user terminal.

In the foregoing specification, the disclosure has been described with reference to specific embodiments. However, as one skilled in the art will appreciate, various embodiments disclosed herein can be modified or otherwise implemented in various other ways without departing from the spirit and scope of the disclosure. Accordingly, this description is to be considered as illustrative and is for the purpose of teaching those skilled in the art the manner of making and using various embodiments of the present application. It is to be understood that the forms of disclosure herein shown and described are to be taken as representative embodiments. Equivalent elements, materials, processes, or steps may be substituted for those representatively illustrated and described herein. Moreover, certain features of the disclosure may be utilized independently of the use of other features, all as would be apparent to one skilled in the art after having the benefit of this description of the disclosure. Expressions such as “including”, “comprising”, “incorporating”, “consisting of”, “have”, “is” used to describe and claim the present disclosure are intended to be construed in a non-exclusive manner, namely allowing for items, components or elements not explicitly described also to be present. Reference to the singular is also to be construed to relate to the plural.

Further, various embodiments disclosed herein are to be taken in the illustrative and explanatory sense, and should in no way be construed as limiting of the present disclosure. All joinder references (e.g., attached, affixed, coupled, connected, and the like) are only used to aid the reader's understanding of the present disclosure, and may not create limitations, particularly as to the position, orientation, or use of the systems and/or methods disclosed herein. Therefore, joinder references, if any, are to be construed broadly. Moreover, such joinder references do not necessarily infer that two elements are directly connected to each other.

Additionally, all numerical terms, such as, but not limited to, “first”, “second”, “third”, “primary”, “secondary”, “main” or any other ordinary and/or numerical terms, should also be taken only as identifiers, to assist the reader's understanding of the various elements, embodiments, variations and/or modifications of the present disclosure, and may not create any limitations, particularly as to the order, or preference, of any element, embodiment, variation and/or modification relative to, or over, another element, embodiment, variation and/or modification.

It will also be appreciated that one or more of the elements depicted in the drawings/figures can also be implemented in a more separated or integrated manner, or even removed or rendered as inoperable in certain cases, as is useful in accordance with a particular application.

What is claimed:

1. A system for sanitizing an enclosed space, the system comprising:
a plurality of sensors configured to:
detect a current environmental condition associated with the enclosed space; and
generate a first sensor data based on the current environmental condition; and
a processor configured to:
obtain a sanitation routine or generate the sanitation routine based at least in part on the first sensor data; and
execute the sanitation routine,
wherein the sanitation routine specifies at least a target environmental condition and a component associated with the enclosed space, and wherein the sanitation routine, when executed by the processor, causes the component to adjust the current environmental condition toward the target environmental condition.

2. The system of claim 1, wherein the plurality of sensors comprise at least one of an image sensor, an acoustic sensor, a thermal sensor, a pressure sensor, a capacitive sensor, a radio frequency sensor or a gas sensor.

3. The system of claim 1, wherein:
the plurality of sensors are further configured to:
detect absence of one or more entities within the enclosed space; and
generate a second sensor data based on the absence of the one or more entities; and
the processor is further configured to:
determine the absence of the one or more entities within the enclosed space based on the second sensor data,
wherein the processor executes the sanitation routine in response to determining the absence of the one or more entities within the enclosed space.

4. The system of claim 1, wherein the current environmental condition comprises an external environmental condition outside of the enclosed space and an internal environmental condition within the enclosed space.

5. The system of claim 1, wherein the enclosed space is a vehicle interior or a building room.

6. The system of claim 1, wherein the component is a light source deployed within the enclosed space and the sanitation routine comprises emitting a visible or an invisible light from the light source.

7. The system of claim 1, wherein the component is a vaporizer deployed within the enclosed space and the sanitation routine comprises emitting vapor from the vaporizer.

8. The system of claim 1, wherein the component is a seat deployed within the enclosed space and the sanitation routine comprises changing a position or an orientation of the seat.

9. The system of claim 1, wherein the component is a seat deployed within the enclosed space and the sanitation routine comprises rolling out a seatbelt of the seat.

10. The system of claim 1, wherein the component is a beating, ventilation, and air conditioning (HVAC) system deployed within the enclosed space, and wherein the sanitation routine comprises increasing a temperature of the enclosed space by the HVAC system.

11. The system of claim 1, wherein the enclosed space is an interior of a vehicle and the component is an autonomous driving system of the vehicle, and wherein the sanitation routine comprises changing an orientation or a position of the vehicle by the autonomous driving system of the vehicle.

12. The system of claim 1, wherein the enclosed space is an interior of a vehicle and the component is a service robot stationed at a parking lot, and wherein the sanitation routine comprises heating the interior of the vehicle by the service robot.

13. The system of claim 1, wherein the enclosed space is an interior of a vehicle and the component is a center display deployed within the interior of the vehicle, and wherein the sanitation routine comprises changing an angle of the center display relative to a light source outside the interior of the vehicle.

14. The system of claim 1, wherein the enclosed space is an interior of a vehicle and the component is a window of the vehicle, and wherein the sanitation routine comprises rolling down the window to expose the interior of the vehicle to a light source outside the interior of the vehicle.

15. A computer-implemented method for sanitizing an enclosed space, wherein the enclosed space comprises a plurality of sensors and a processor, the method comprising:
detecting, by the plurality of sensors, a current environmental condition associated with the enclosed space;
generating, by the plurality of sensors, a first sensor data based on the current environmental condition;
receiving or generating, by the processor, a sanitation routine, wherein the sanitation routine is generated based at least in part on the first sensor data; and
causing, by the processor, a component associated with the enclosed space to adjust the current environmental condition associated with the enclosed space toward a target environmental condition,
wherein the sanitation routine specifies at least the target environmental condition and the component associated with the enclosed space.

16. The computer-implemented method of claim 15, further comprising:
detecting, by the plurality of sensors, absence of one or more entities within the enclosed space;
generating, by the plurality of sensors, a second sensor data based on the absence of the one or more entities; and
determining, by the processor, the absence of the one or more entities within the enclosed space based on the second sensor data,
wherein the processor executes the sanitation routine in response to determining the absence of the one or more entities within the enclosed space.

17. The computer-implemented method of claim 16, further comprising:
receiving, by the processor, weather information from a source external to the enclosed space, and wherein the processor generates the sanitation routine further based on the weather information in response to determining the absence of the one or more entities within the enclosed space.

18. One or more non-transitory computer-readable media comprising instructions executable at a sanitation system of an enclosed space, wherein the instructions, when executed by the sanitation system, cause the sanitation system to:
detect a current environmental condition associated with the enclosed space;
generate a first sensor data based on the current environmental condition;
generate a sanitation routine based at least in part on the first sensor data; and
execute the sanitation routine,
wherein the sanitation routine specifies at least a target environmental condition and a component associated with the enclosed space, and wherein executing the sanitation routine causes the component to adjust the current environmental condition toward the target environmental condition.

19. The one or more non-transitory computer-readable media of claim 18, wherein the current environmental condition comprises an external environmental condition outside of the enclosed space and an internal environmental condition within the enclosed space.

20. The one or more non-transitory computer-readable media of claim 18, wherein the enclosed space is a vehicle interior or a building room.
